(19) United States
(12) Patent Application Publication
(10) Pub. No.: US 2014/0057940 A1
(43) Pub. Date: Feb. 27, 2014

(54) Methods for Treating or Preventing Cardiac and Neurological Disorders Using Chemokine Receptor Antagonists

(71) Applicants: Joseph L. Mankowski, Severna Park, MD (US); David R. Graham, Arbutus, MD (US)

(72) Inventors: Joseph L. Mankowski, Severna Park, MD (US); David R. Graham, Arbutus, MD (US)

(21) Appl. No.: 13/979,085

(22) PCT Filed: Jan. 11, 2012
(86) PCT No.: PCT/US12/20938

§ 371 Date: Oct. 30, 2013

Related U.S. Application Data
(60) Provisional application No. 61/431,555, filed on Jan. 11, 2011.
(60) Provisional application No. 61/437,241, filed on Jan. 28, 2011.

(51) Int. Cl. A61K 31/46

(52) U.S. Cl. CPC A61K 31/46
USPC 514/304

(57) ABSTRACT

The invention provides methods for treating or preventing cardiac and neurological disorders using chemokine receptor antagonists.

### CROSS-REFERENCE TO RELATED APPLICATIONS

This application claims the benefit of U.S. Provisional Application No. 61/431,555, filed on Jan. 11, 2011, and U.S. Provisional Application No. 61/437,241, filed on Jan. 28, 2011; the contents of each of which is specifically incorporated by reference herein in its entirety.

### STATEMENT OF RIGHTS

This invention was made with government support under Grant RO1 HL078479 awarded by the National Institutes of Health. The U.S. government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

Primate immunodeficiency virus (PIV) infection has traditionally been associated with immunological deficiencies associated with acquired immunodeficiency syndrome (AIDS). However, PIV infection also manifests a number of clinically distinct conditions, such as cardiac dysfunction and neurological disorders, which themselves have a large impact on human health. For example, despite major developments in both diagnosis and treatment, cardiac-related disorders, such as heart failure (HF), continue to be a leading cause of death and disability in older adults worldwide and affect more than 12 million patients in the United States and Europe alone (Lloyd-Jones et al. (2010) Circulation 121:948-954). To date, however, knowledge of the molecular and cellular mediators of the PIV infection-related cardiac dysfunction and neurological disorders remains limited. Accordingly, there exists a need in the art to identify specific molecular targets that can be used to diagnose, prognose, and treat such disorders in patients regardless of PIV infection status.

### SUMMARY OF THE INVENTION

The present invention is based, at least in part, on the discovery that chemokine receptors, such as CCR5, are expressed by primate cardiomyocytes and neurons and that their activation triggers signaling pathways that impair cardiac and neurological function independent of infection of the cells by primate immunodeficiency viruses. In addition, it has been demonstrated that blocking signaling by such chemokine receptors using chemokine receptor antagonists can treat and/or prevent the cardiac and neurological disorders from arising and independently of infection of the cells by primate immunodeficiency viruses.

In one aspect, the present invention provides a method of treating or preventing a cardiac disorder in a subject comprising administering to the subject an effective amount of at least one antagonist of a chemokine receptor expressed by cardiomyocytes and/or inflammatory cells in the myocardium of the subject, wherein binding of the chemokine receptor antagonist reduces binding of at least one chemokine to the chemokine receptor, thereby treating or preventing the cardiac disorder in the subject.

In another aspect, the present invention provides a method of treating or preventing a neurological disorder in a subject comprising administering to the subject an effective amount of at least one antagonist of a chemokine receptor expressed by neurons of the subject, wherein binding of the chemokine receptor antagonist reduces binding of at least one chemokine to the chemokine receptor, thereby treating or preventing the neurological disorder in the subject.

### BRIEF DESCRIPTION OF FIGURES

FIGS. 1A-1E show CCL5 decreases myocardial contractility without altering calcium flux. FIG. 1A shows sarcomere length as a function of time in isolated macaque cardiomyocytes either unexposed or exposed to maraviroc (n=4 single cardiomyocyte recordings). FIG. 1B shows a representative single twitch trace of sarcomeric contraction. FIG. 1C shows the mean percentage decline in sarcomere contraction induced by CCL5 in either the presence or absence of maraviroc exposure. FIG. 1D shows that CCL5 significantly decreases sarcomeric shortening (p<0.001, paired t-test comparing recordings from 14 VCM, mean of 20 cell contraction cycles/cell at steady state), which is reversed by exposure to maraviroc. FIG. 1E shows calcium transient amplitudes was not altered by CCL5 (RANTES) or CCL5 (RANTES)+MVC treatment (ANOVA P=0.72).

FIG. 2 is a schematic illustration of the signal transduction and effector pathway by which SIV and chemokine ligands alter cardiomyocyte contractility.

FIG. 3 shows identification, gender, SIV inoculation age, SIVE, and days post inoculation at euthanasia data for the macaque subjects described in Examples 1-3.

FIG. 4 shows cerebrospinal fluid (CSF) and plasma bioavailability of maraviroc in macaque subjects as a function of time after single dose oral administration.

FIG. 5 shows survival, CD4⁺ cell count, CSF viral load, plasma viral load, spleen viral load, and heart viral load data among macaque subjects.

FIG. 6 shows additional CSF viral load, plasma viral load, spleen viral load, and heart viral load data among macaque subjects. Untreated SIV animals are subdivided into those with rapid disease progression (RP) and those with long term progression (LTP; over 180 days post-SIV inoculation). Maraviroc treatment improves outcomes in both RP and LTP groups, indicating maraviroc efficacy even in long term progression subjects.

FIG. 7 shows relative changes in mitral valve early diastolic wave time (MV E), mitral valve ratio of transmitral early to late peak velocities (MV E/A), mitral valve isovolumetric relaxation time (MV IVRT), mitral valve deceleration time (MV DT), lateral isovolumetric relaxation time (lateral IVRT), and ejection fraction percentage (EF) among macaque subjects obtained by echocardiography.

FIG. 8 shows data regarding immunostaining for CD68⁺ macrophages and CD 163 expression by resident and infiltrating macrophages in the myocardium of macaque subjects. CD58 levels do not change according to maraviroc treatment, whereas CD163 expression is lowered to levels present in uninfected and untreated subjects.

FIG. 9 shows myocardial viral load data among macaque subjects.

FIG. 10 shows basal ganglia viral load, CD68⁺ macrophage immunostaining, β-amyloid precursor protein (APP) immunostaining in axons, and general viral load data among macaque subjects.

### DETAILED DESCRIPTION OF THE INVENTION

The present invention is based in part on the elucidation of methods and mechanisms useful for treating or preventing cardiac and neurological disorders using chemokine receptor antagonists.

In one embodiment, the subject is infected with at least one primate immunodeficiency virus (PIV), such as simian immunodeficiency virus (SW), human immunodeficiency virus 1 (HIV-1), or human immunodeficiency virus 2 (HIV-2). In another embodiment, the subject is not infected with a PIV. In still another embodiment, the chemokine receptor is selected from the group consisting of chemokine receptor 5 (CCR5), a receptor that can bind chemokine ligand CCL3, a receptor that can bind chemokine ligand CCL4, a receptor that can bind chemokine ligand CCL5, a chemokine receptor that can bind SIV, a chemokine receptor that can bind HIV-1, and a chemokine receptor that can bind HIV-2. In yet another embodiment, the chemokine receptor antagonist is selected from the group consisting of a nucleic acid, a peptide, a peptidomimetic, an antibody, and a small molecule that binds the chemokine receptor or nucleic acid encoding the chemokine receptor. For example, the chemokine receptor antagonist can be a small molecule selected from the group consisting of 4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide, vicriviroc, NCB-9471, PRO-140, CCR5 mAb004, 8-[4-(2-butoxyethoxy)phenyl]-1-isobutyl-N-[4-[[(1-propyl-1H-imadazol-5-yl-)methyl]sulphinyl]phenyl]-1,2,3,4-tetrahydro-1-benzacocine-5-carboxamide, methyl1--endo-{8-[(3S)-3-(acetylamino)-3-(3-fluorophenyl)propyl]-8-azabicy-[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carbox-ylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carb-oxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide), Sch-C, Sch-D, TAK-220, PRO-140, or a pharmaceutically acceptable salt or solvate thereof. In another embodiment, the subject is human.

In still another embodiment, the cardiac disorder is selected from the group consisting of myocarditis, dilated cardiomyopathy, left ventricular dysfunction, atherosclerosis, coronary artery disease, coronary heart disease, coronary vascular disease, peripheral vascular disease, myocardial infarction, and heart failure. In yet another embodiment, the cardiac disorder is selected from the group consisting of a) decreased cardiomyocyte contractility; b) decreased left ventricular ejection fraction and/or volume; c) increased end-systolic and/or end-diastolic fractional shortening; d) decreased mitral valve annular velocity; e) decreased mitral inflow; f) decreased aortic velocity time integral; g) decreased aorta cross section area; h) decreased isovolumetric contraction time; i) increased isovolumetric relaxation time; j) decreased heart chamber mechanical efficiency; k) increased cardiac protein kinase A and/or protein kinase C biomarker expression or activity; l) decreased phosphorylation of cardiac myofilament protein biomarker; m) increased macrophage activation and/or infiltration in the myocardium; n) increased chemokine biomarker expression and/or activity in the myocardium; o) increased cytokine biomarker expression and/or activity in the myocardium; p) increased cardiac fibrosis; q) increased cardiac inflammation; and r) increased ventricular dilation.

In yet another embodiment, the cardiac disorder is determined by assessing a) cardiomyocyte contractility; b) left ventricular ejection fraction and/or volume; c) end-systolic and/or end-diastolic fractional shortening; d) mitral valve annular velocity; e) mitral inflow; f) aortic velocity time integral; g) aorta cross section area; h) isovolumetric contraction time; i) isovolumetric relaxation time; j) heart chamber mechanical efficiency; k) cardiac protein kinase A and/or protein kinase C biomarker expression or activity; l) phosphorylation of a cardiac myofilament protein biomarker; m) macrophage activation and/or infiltration in the myocardium; n) chemokine biomarker expression and/or activity in the myocardium; o) cytokine biomarker expression and/or activity in the myocardium; p) cardiac fibrosis; q) cardiac inflammation; or r) ventricular dilation in the subject or relative to a baseline. In one embodiment, the expression of the biomarker is assessed by detecting the presence in the sample of a protein corresponding to the biomarker. In another embodiment, the presence of the protein is detected using a reagent which specifically binds with the protein. In still another embodiment, the reagent is selected from the group consisting of an antibody, an antibody derivative, and an antibody fragment. In yet another embodiment, the expression of the biomarker is assessed by detecting the presence of a transcribed polynucleotide or portion thereof, wherein the transcribed polynucleotide comprises the biomarker (e.g., mRNA or a cDNA). In another embodiment, the step of detecting further comprises amplifying the transcribed polynucleotide. In still another embodiment, the chemokine receptor antagonist is administered in a pharmaceutically acceptable formulation. In yet another embodiment, the method further comprises administering one or more agents that inhibit the cardiac disorder, such as a protease inhibitor, an integrase inhibitor, a nucleoside reverse transcriptase inhibitor, and/or a nucleotide reverse transcriptase inhibitor.

In still another embodiment, the neurological disorder is selected from the group consisting of Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy(LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders. In yet another embodiment, the neurological disorder is selected from the group consisting of a) increased macrophage activation and/or infiltration in the central nervous system (CNS) or peripheral nervous system (PNS); b) increased amyloid precursor protein biomarker expression and/or activity in the CNS or PNS; c) increased lesion formation in the CNS; d) decreased neurite growth in the CNS or PNS; e) increased neuronal cell death in the CNS or PNS; and f) increased neural inflammation in the CNS or PNS.

In yet another embodiment, the neurological disorder is determined by assessing a) macrophage activation and/or infiltration in the central nervous system (CNS) or peripheral nervous system (PNS); b) amyloid precursor protein biomarker expression and/or activity in the CNS or PNS; c) lesion formation in the CNS; d) neurite growth in the CNS or PNS; e) neuronal cell death in the CNS or PNS; or f) neural inflammation in the CNS or PNS in the subject or relative to a baseline.

### I. DEFINITIONS

In order that the present invention can be more readily understood, certain terms are first defined. Additional definitions are set forth throughout the detailed description.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

Unless otherwise specified herein, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives can comprise a protein or chemical moiety conjugated to an antibody. The term “antibody” as used herein also includes an “antigen-binding portion” of an antibody (or simply “antibody portion”). The term “antigen-binding portion,” as used herein, refers to one or more fragments of an antibody that retain the ability to specifically bind to an antigen (e.g., chemokine ligand and/or chemokine receptor polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Still further, an antibody or antigen-binding portion thereof can be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein. Antibodies can be polyclonal or monoclonal; xenogeneic, allogeneic, or syngeneic; or modified forms thereof (e.g., humanized, chimeric, etc.). Antibodies can also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “binding” or “interacting” refers to an association, which can be a stable association, between two molecules, e.g., between a polypeptide of the invention and a binding partner, due to, for example, electrostatic, hydrophobic, ionic and/or hydrogen-bond interactions under physiological conditions. Exemplary interactions include protein-protein, protein-nucleic acid, protein-small molecule, and small molecule-nucleic acid interactions.

The term “biological sample” is intended to include tissues, cells and biological fluids isolated from a subject, as well as tissues, cells and fluids present within a subject.

The term “body fluid” refers to fluids that are excreted or secreted from the body as well as fluid that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, and vomit). In some embodiments, media described herein can contain or comprise body fluids.

The term “cardiac contractility” or “myocardial contractility” refer to measures of cardiac function, which may include but are not limited to cardiac output, ejection fraction, fractional shortening, cardiac work, cardiac index, chronotropy, lusitropy, velocity of circumferential fiber shortening, velocity of circumferential fiber shortening corrected for heart rate, stroke volume, rates of cardiac contraction or relaxation, the first derivatives of interventricular pressure (maximum dP/dt and minimum dP/dt), ventricular volumes, clinical evaluations of cardiac function (for example, stress echocardiography and treadmill walking) and variations or normalizations of these parameters. These parameters may be measured in humans or animals alike to assess myocardial function and assist in diagnosis and prognosis of cardiac-related disorders.

The term “cardiac disorder” refers to any disorder or condition involving cardiac muscle tissue or cardiac dysfunction. Disorders involving cardiac muscle tissue include, but are not limited to, myocardial disease, including but not limited to dilated cardiomyopathy, hypertrophic cardiomyopathy, restrictive cardiomyopathy, myocardial stunning, and myocarditis; heart failure; acute heart failure; rheumatic fever; rhabdomyoma; sarcoma; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia; disorders involving cardiac transplantation; arterial hypertension; peripartum cardiomyopathy; alcoholic cardiomyopathy; tachycardias; supraventricular tachycardia; bradycardia; atrial flutter; hydrops fetalis; arrhythmias; extrasystolic arrhythmia; fetal cardiac arrhythmia; endocarditis; atrial fibrillation; idiopathic dilated cardiomyopathy; Chagas' heart disease; long QT syndrome; Brugada syndrome; ischemia; hypoxia; ventricular fibrillation; ventricular tachycardia; restenosis; congestive heart failure; syncope; arrythmias; pericardial disease; myocardial infarction; unstable angina; stable angina; and angina pectoris, viral myocarditis, and non-proliferating cell disorders involving cardiac muscle tissue.

The term “chemokine” or “chemokine ligand” refers to a family of chemotactic cytokines approximately 8-10 kDa in size, that are released by a wide variety of cells, to attract macrophages, T cells, eosinophils, basophils, and neutrophils to sites of inflammation and also play a role in the maturation of cells of the immune system (see, for example, Ponath (1998) Exp. Opin. Invest. Drugs 7:1-18). Chemokines appear to share a common structural motif that consists of 4 conserved cysteines involved in maintaining tertiary structure. There are two major subfamilies of chemokines: the “CC” or beta-chemokines and the “CXC” or alpha-chemokines, depending on whether the first two cysteines are separated by a single amino acid, i.e., CXC or are adjacent, i.e., CC. These chemokines bind specifically to cell-surface receptors belonging to the family of G-protein-coupled seven-transmembrane proteins which are referred to as “chemokine receptors”, and mediate biological activity through these receptors. Chemokines are considered to be principal mediators in the initiation and maintenance of inflammation (see Chemokines in Disease published by Humana Press (1999), Edited by C. Herbert; Murdoch et al. Blood 95, 3032-3043 (2000)). More specifically, chemokines have been found to play an important role in the regulation of endothelial cell function, including proliferation, migration and differentiation during angiogenesis and re-endothelialization after injury (Gupta et al. (1998) J. Biol. Chem. 7:4282-4287). In particular, the term “CCL3” refers to a specific chemokine ligand. The sequence of human CCL3 is available to the public at the GenBank database under NM_{—}002983.2 and NP_{—}002974.1. Nucleic acid and polypeptide sequences of CCL3 orthologs in organisms other than humans are well known and include, for example, monkey CCL3 (NM_{—}001034200.1 and NP_{—}001029372.1), chimpanzee CCL3 (NM_{—}001034082.1 and NP_{—}001029254.1), dog CCL3 (NM_{—}001253735.1 and NP_{—}001240664.1), and cow CCL3 (XM_{—}002695625.1 and XP_{—}002695671.1). In addition, the term “CCL4” refers to a specific chemokine ligand. The sequence of human CCL4 is available to the public at the GenBank database under NM_{—}002984.2 and NP_{—}002975.1. Nucleic acid and polypeptide sequences of CCL4 orthologs in organisms other than humans are well known and include, for example, monkey CCL4 (NM_{—}001032873.1 and NP_{—}001028045.1), dog CCL4 (NM_{—}001005250.1 and NP_{—}001005250.1), cow CCL4 (NM_{—}001075147.1 and NP_{—}001068615.1), mouse CCL4 (NM_{—}013652.2 and NP_{—}038680.1), and rat CCL4 (NM_{—}053858.1 and NP_{—}446310.1). Moreover, the term “CCL5” refers to a specific chemokine ligand. The sequence of human CCL5 is available to the public at the GenBank database under NM_{—}002985.2 and NP_{—}002976.2. Nucleic acid and polypeptide sequences of CCL5 orthologs in organisms other than humans are well known and include, for example, monkey CCL5 (NM_{—}001032850.1 and NP_{—}001028022.1), chimpanzee CCL5 (XM_{—}001155572.2 and XP_{—}001155572.2), dog CCL5 (NM_{—}001003010.1 and NP_{—}001003010.1), cow CCL5 (NM_{—}175827.2 and NP_{—}787021.1), mouse CCL5 (NM_{—}013653.3 and NP_{—}038681.2), rat CCL5 (NM_{—}031116.3 and NP_{—}112378.2), and chicken CCL5 (NM_{—}001045832.1 and NP_{—}001039297.1).

The term “chemokine receptor” refers to a family of natural binding partners of for chemokines. Chemokine receptors of the beta-chemokines are designated “CCR”; while those of the alpha-chemokines are designated “CXCR.” These chemokine receptors include, but are not limited to CCR1, CCR2, CCR2A, CCR2B, CCR3, CCR4, CCR5, CCR6, CXCR3 and CXCR4 (see, for example, Murphy et al. (2000) Pharmacol. Rev. 52:145-176). In particular, the term “CCR5” refers to a specific chemokine receptor family member. At least two human transcript variants encoding the same human CCR5 proteins exist. The sequence of human CCR5 variant A is available to the public at the GenBank database under NM_{—}000579.3 and NP_{—}000570.1. The sequence of human CCR5 transcript variant B is longer than that of variant A due to a different 5′ untranslated region and is publicly available under NM_{—}001100168.1 and NP_{—}001093638.1. Nucleic acid and polypeptide sequences of CCR5 orthologs in organisms other than humans are well known and include, for example, monkey CCR5 (NM_{—}001042773.2 and NP_{—}001036238.2), mouse CCR5 (NM_{—}009917.5 and NP_{—}034047.2), chimpanzee CCR5 (NM_{—}001009046.1 and NP_{—}001009046.1), dog CCR5 (NM_{—}001012342.2 and NP_{—}001012342.2), cow CCR5 (NM_{—}0010116572.2 and NP_{—}00101672.2), rat CCR5 (NM_{—}053960.3 and NP_{—}446412.2), and chicken CCR5 (NM_{—}001045834.1 and NP_{—}001039299.1). In addition, the term “CXCR4” or “C—X—C chemokine receptor type 4” refers to a chemokine receptor which binds members of the C—X—C group of chemokines. At least two transcript variants encoding the same human CXCR4 protein exist. The sequence of human CXCR4 transcript variant 1, which encodes the longer of the two human CXCR4 isoforms (i.e., isoform a), is available to the public at the GenBank database under NM_{—}001008540.1 and NP_{—}001008540.1. The sequence of human CXCR4 transcript variant 2 differs in the 5′ untranslated region (UTR) and lacks an in-frame portion of the 5′ coding region compared to variant 1 and therefore encodes a smaller polypeptide having a shorter N-terminus relative to that of isoform 1. Such nucleic acid and protein sequences can be found under NM_{—}003467.2 and NP_{—}003458.1. Nucleic acid and polypeptide sequences of CXCR4 orthologs in organisms other than humans are well known and include, for example, mouse CXCR4 (NM_{—}009911.3 and NP_{—}034041.2), chimpanzee CXCR4 (NM_{—}001009047.1 and NP_{—}001009047.1), rat CXCR4 (NM_{—}022205.3 and NP_{—}071541.2), cow CXCR4 (NM_{—}174301.3 and NP_{—}776726.1), dog CXCR4 (NM_{—}001048026.1 and NP_{—}001041491.1), and chicken CXCR4 (NM 204617.2 and NP 989948.2). As used herein, CXCR4 includes extracellular portions of CXCR4 capable of binding the PIV envelope protein.

The term “chemokine receptor antagonist” refers to any agent capable of reducing the amount and/or activity of the chemokine receptor. Exemplary embodiments include a CCR1 receptor antagonist, CCR4 receptor antagonist, CCR5 receptor antagonist, CXCR3 receptor antagonist, CCR3 receptor antagonist, CCR2 receptor, CX3CR1 receptor antagonist, CXCR4 receptor antagonist, or CD4 receptor antagonist. In other embodiments, chemokine receptor antagonists include at least one antagonist of one or more of CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3. In some embodiments, the antagonism can primarily be measured according to inhibition of activity according to the methods described herein, according to binding affinity (e.g., an IC₅₀ for CCR5 binding of less than 1 μM as determined by the MIP-1beta assay of Combadiere et al, J. Leukoc. Biol., 60, 147-152 (1996), or a combination of both. In addition, many small molecule chemokine receptor antagonists are known in the art. For example, CCR5 antagonists include aplaviroc, nifeviroc, vicriviroc (SCH-417690), maraviroc (Selzentry), PRO-140, PRO-542, INCB15050, INCB9471, PF-232798, SCH-532706, GSK-706769, TAK-652, TAK-220, ESN-196, RO-1752, ZM-688523, AMD-887, YM-370749, NIBR-1282, SCH-350634, and ZM-688523.

The term “complex” refers to an association between at least two moieties (e.g. chemical or biochemical) that have an affinity for one another. “Protein complex” or “polypeptide complex” refers to a complex comprising at least one or more polypeptides.

The term “effective amount” refers to an amount sufficient to achieve a desired result. For example, a “prophylactically effective amount” refers to an amount sufficient to reduce the likelihood of a disorder from occurring. In addition, a “therapeutically effective amount” refers to an amount effective to slow, stop or reverse the progression of a disorder.

The term “exposure” to a PIV refers to contact with the PIV such that infection could result.

The term “heart cell” refers to a cell which can be: (a) part of a heart present in a subject, (b) part of a heart which is maintained ex vivo, (c) part of a heart tissue, or (d) a cell which is isolated from the heart of a subject. For example, the cell can be a muscle cell, such as a cardiac myocyte (cardiomyocyte) or smooth muscle cell. Heart cells of the invention can also include endothelial cells within the heart, for example, cells of a capillary, artery, or other vessel.

The term “heart failure” generally refers to any disorder in which the heart has a defect in its ability to pump adequately to meet the body's needs. In many cases, heart failure is the result of one or more abnormalities at the cellular level in the various steps of excitation-contraction coupling of the cardiac cells. It is most frequently due to a defect in myocardial contraction, which can occur for many reasons, the most common of which include: ischemic damage to the myocardium, excessive mechanical resistance to the outflow of blood from the heart, overloading of the cardiac chambers due to defective valve function, infection or inflammation of the myocardium, or congenitally poor myocardial contractile function (Braunwald (2001) Harrison's Principles of Internal Medicine, 15th ed., pp 1318-29).

The term “inhibit” includes the decrease, limitation, or blockage, of, for example a particular action, function, or interaction. For example, a cardiac-related disorder is “inhibited” if at least one symptom of the disease, such as reduced mechanical efficiency, is alleviated, terminated, slowed, or prevented. As used herein, a cardiac-related disorder is also “inhibited” if recurrence of a disease symptom is reduced, slowed, delayed, or prevented. Such an inhibition can affect a cardiac-mediated activity (e.g., blood pressure, blood flow rates, and the like). For example, a cardiac-mediated activity can be decreased by 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more or any range in between. The terms “increase,” “enhance,” “promote,” and the like can be used in the exact opposite manner as “inhibit.”

The term “substantially altered,” “substantially modulated,” and the like, unless otherwise defined, refers to a deviation of a measured attribute in comparison to a reference control. The deviation can be measured according to quantitative or qualitative means. In one embodiment, the attribute's alteration is greater than 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or more or any range in between different relative to the control.

The terms “label” or “labeled” refer to incorporation or attachment, optionally covalently or non-covalently, of a detectable marker into a molecule, such as a polypeptide. Various methods of labeling polypeptides are known in the art and can be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

The term “maraviroc” refers to a small molecule CCR5 antagonist developed by Pfizer, Inc. and is also known under the trade names “Selzentry” and UK-427857. The chemical structure of maraviroc is 4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide. The definition encompasses derivatives, salts, solvent forms, and the like having the ability to antagonize CCR5, including, but not limited to, vicriviroc, NCB-9471, PRO-140, CCR5 mAb004, 8-[4-(2-butoxyethoxy)phenyl]-1-isobutyl-N-[4-[[(1-propyl-1H-imadazol-5-yl-) methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicy-[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carbox-ylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carb-oxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide).

An “overexpression” or “significantly higher level of expression or copy number” of a marker refers to an expression level or copy number in a test sample that is greater than the standard error of the assay employed to assess expression or copy number, and is preferably at least twice, and more preferably three, four, five or ten or more times the expression level or copy number of the marker in a control sample (e.g., sample from a healthy subject not afflicted with a cardiac-related disorder) and preferably, the average expression level or copy number of the marker in several control samples.

The term “pharmaceutically acceptable salts” includes the salts of compounds that are, within the scope of sound medical judgment, suitable for use with patients without undue toxicity, irritation, allergic response, and the like, commensurate with a reasonable benefit/risk ratio, and effective for their intended use, as well as the zwitterionic forms, where possible, of the compounds.

The term “primate immunodeficiency virus” or “PIV” refers to a group of well-known viruses infecting primates. The term includes human immunodeficiency viruses (HIV) and simian immunodeficiency viruses (SIV). For example, the term includes the human viruses, HIV-1 and HIV-2; the chimpanzee virus SIVcpz such as, for example, SIVcpzGab, SIVcpzCam, SIVcpzAnt, and SIVcpzUS; the sooty mangabey virus SIVsm; the African green monkey virus SIVagm such as, for example, SIVagm-1 and SIVagm-2; the mandrill virus SIVmnd such as, for example, SIVmnd14 and SIV mndGB 1, as well as a host of others including SIVsun/lhoest, SIVcol, SIVrcm, SIVsyk, SIVdeb, SIVgsn, SIVmon, SIVmus, and SIVtal. PIV is inclusive of all strains (e.g., SIVcpz) and sub-strains (e.g., SIVcpzGab). Regarding human immunodeficiency viruses, HIVs can be categorized into multiple clades with a high degree of genetic divergence. As used herein, the term “clade” refers to related human immunodeficiency viruses classified according to their degree of genetic similarity. There are currently three groups of HIV-1 isolates: M, N, and O. Group M (major strains) consists of at least ten clades, A through J. Group 0 (outer strains) can consist of a similar number of clades. Group N is a new HIV-1 isolate that has not been categorized in either group M or O.

The term “response to therapy” relates to any response of the cardiac-related disorder to a therapy. Responses can be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of cardiac-related disorder response can be done early after the onset of therapy, e.g., after a few hours, days, weeks or preferably after a few months. Additional criteria for evaluating the response to therapies are related to “survival,” which includes all of the following: survival until mortality, also known as overall survival (wherein said mortality can be either irrespective of cause or tumor related); “recurrence-free survival” (e.g., viral load below a detectable threshold); metastasis free survival; disease free survival. The length of said survival can be calculated by reference to a defined start point (e.g., time of diagnosis or start of treatment) and end point (e.g., death, recurrence or metastasis). In addition, criteria for efficacy of treatment can be expanded to include response to therapy, probability of survival, probability of disease manifestation recurrence within a given time period. For example, in order to determine appropriate threshold values, a particular therapeutic regimen can be administered to a population of subjects and the outcome can be correlated to viral load or other measurements that were determined prior to administration of any therapy. Alternatively, outcome measures, such as overall survival and disease-free survival can be monitored over a period of time for subjects following therapy for whom measurement values are known.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a subject having or at risk for having cancer, to inhibit expression of a marker gene of the invention, e.g., a marker gene which is overexpressed in cancer (such as the markers listed in Table 3) and thereby treat, prevent, or inhibit cancer in the subject.

The term “subject” refers to any healthy animal, mammal or human, or any animal, mammal or human afflicted with a cardiac-related disorder.

As used herein, the term “survival” includes all of the following: survival until mortality, also known as overall survival (wherein said mortality can be either irrespective of cause or tumor related); “recurrence-free survival” (wherein the term recurrence shall include both localized and distant recurrence); disease free survival (wherein the term disease shall include antiviral infection and diseases associated therewith). The length of said survival can be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of disease recurrence. In some embodiments, mortality is measured in terms of survival rates.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter. For example, quantitative or tissue specificity upstream elements from cardiac-specific promoters may be used to express a gene product in cardiac tissue or cells thereof. Many such elements have been characterized and include, without limitation, the murine TIMP-4 promoter, A and B-type natriuretic peptide promoters, human cardiac troponin I promoter, murine S100A1 promoter, salmon cardiac peptide promoter, GATA response element, rabbit β-myosin promoter, and mouse α-myosin heavy chain promoter (Rahkonen, et al. (2002) Biochem Biophys Acta 1577:45-52; Thuerauf and Glembotski (1997) J. Biol. Chem. 272:7464-7472; LaPointe et al (1996) Hypertension 27:715-722; Grepin et al. (1994) Mol. Cell. Biol. 14:3115-29; Dellow, et al. (2001) Cardiovasc. Res. 50:3-6; Kiewitz, et al. (2000) Biochem Biophys Acta 1498:207-19; Majalahti-Palviainen, et al (2000) Endocrinology 141:731-740; Charron et al. (1999) Molecular & Cellular Biology 19:4355-4365; Genbank 071441; U.S. Provisional Patent Application No. 60/393,525 and 60/454,947; and U.S. patent application Ser. No. 10/613,728). In addition, cardiac tissue preferred promoter elements from the following genes can be used: myosin light chain-2, .alpha.-myosin heavy chain, AE3, cardiac troponin C, and cardiac .alpha.-actin. See, e.g. Franz et al (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol Cell Biol. 14:1870-1885; Hunter et al. (1993) Hypertension 22:608-617; and Sartorelli et al. (1992) Proc. Natl. Acad. Sci. USA 89:4047-4051. In some embodiments, the coding region is operably linked to an inducible regulatory element or elements. A variety of inducible promoter systems has been described in the literature and can be used in the present invention. A known and useful conditional system is the binary, tetracycline-based system, which has been used in both cells and animals to reversibly induce expression by the addition or removal of tetracycline or its analogues. Another example of such a binary system is the cre/loxP recombinase system of bacteriophage P1. For a description of the cre/loxP recombinase system, see, e.g., Lakso et al. (1992) PNAS 89:6232-6236. Another class of promoter elements are those which activate transcription of an operably linked nucleotide sequence of interest in response to hypoxic conditions. These include promoter elements regulated at least in part by hypoxia inducible factor-1. Hypoxia response elements include, but are not limited to, the erythropoietin hypoxia response enhancer element (HREE1), the muscle pyruvate kinase HRE; the beta-enolase HRE; and endothelin-1 HRE element, and chimeric nucleotide sequence comprising these sequences. See Bunn and Poynton (1996) Physiol. Rev. 76:839-885; Dachs and Stratford (1996) Br. J. Cancer 74:S126-S132; Guillemon and Krasnow (1997) Cell 89:9-12; Firth et al. (1994) Proc. Natl. Acad. Sci. 91:6496-6500; Jiang et al. (1997) Cancer Res. 57:5328-5335; U.S. Pat. No. 5,834,306). It is further recognized that to increase transcription levels or to alter tissue specificity, enhancers and/or tissue-preference elements may be utilized in combination with a given promoter.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cardiac-related disorder) and preferably, the average expression level or copy number of the marker in several control samples.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid | GAC, GAT |
| (Asp, D) | |
| Cysteine (Cys, C) | TGC, TGT |
| Glutamic acid | GAA, GAG |
| (Glu, E) | |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine | TTC, TTT |
| (Phe, F) | |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet can be employed (illustrated above). Therefore, a number of different nucleotide sequences can code for a given amino acid sequence. Such nucleotide sequences are considered functionally equivalent since they result in the production of the same amino acid sequence in all organisms (although certain organisms can translate some sequences more efficiently than they do others). Moreover, occasionally, a methylated variant of a purine or pyrimidine can be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Before the present invention is further described, it will be appreciated that specific sequence identifiers (SEQ ID NOs) have been referenced throughout the specification for purposes of illustration and should therefore not be construed to be limiting. Any marker of the invention, including, but not limited to, the markers described in the specification and markers described herein are well known in the art and can be used in the embodiments of the invention.

It is further to be understood that this invention is not limited to particular embodiments described, as such can, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an chemokine ligand-chemokine receptor complex” includes a plurality of such complexes and reference to “the active agent” includes reference to one or more active agents and equivalents thereof known to those skilled in the art, and so forth. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

### II. COMPOSITIONS OF THE INVENTION

Some embodiments of the present invention are directed to methods using chemokine receptors, chemokine ligands, and chemokine receptor antagonists. In certain embodiments, such compositions are encoded by nucleic acid molecules and/or comprise polypeptides. Nucleic acid sequences described herein and/or well known in the art can be altered or designed to encode the same or a substantially similar amino acid sequence or protein having the desired biological activities. In addition, the amino acid sequences or proteins described herein can be altered or modified according to methods known in the art to have different sequences yet still be capable of exhibiting the desired biological activities. It is to be understood that the specific amino acid sequences and proteins described herein include sequences and proteins that are substantially similar or homologous thereto or those that can be modified in a manner contemplated by those skilled in the art without departing from the spirit and operation of the present invention. The following are representative examples of chemokine ligand and chemokine receptor sequences:

| Human CCL3 coding nucleic acid sequence (NCBI Accession NM_002983.2): | |
|---|---|
| 1 | atgcaggtct ccactgctgc ccttgctgtc ctcctctgca ccatggctct |
| | ctgcaaccag |
| 61 | ttctctgcat cacttgctgc tgacacgccg accgcctgct gcttcagcta |
| | cacctcccgg |
| 121 | cagattccac agaatttcat agctgactac tttgagacga gcagccagtg |
| | ctccaagccc |
| 181 | ggtgtcatct tcctaaccaa gcgaagccgg caggtctgtg ctgaccccag |
| | tgaggagtgg |
| 241 | gtccagaaat atgtcagcga cctggagctg agtgcctga |
| Human CCL3 protein sequence (NCBI Accession NP_002974.1): | |
| 1 | mqvstaalav llctmalcnq fsaslaadtp taccfsytsr qipqnfiady |
| | fetssqcskp |
| 61 | gvifltkrsr qvcadpseew vqkyvsdlel sa |
| Monkey CCL3 coding nucleic acid sequence (NCBI Accession NM_001034200.1): | |
| 1 | atgcaggtct ccactgctgc ccttgctgtc ctcctctgca ccgtggctct |
| | ctgcaaccgg |
| 61 | atctcagcaa catttgctgc tgacaccccg acctcctgct gcttcagcta |
| | catctcccgg |
| 121 | cagattccac agaatttcat agctgactac tttgagacca acagccagtg |
| | ctccaagccc |
| 181 | ggtgtcatct tcctaaccaa gagaggccgg caggtctgtg ctgaccccag |
| | taaggagtgg |
| 241 | gtccagaaat acgtcagcga cctagagctg agtgcctga |
| Monkey CCL3 protein sequence (NCBI Accession NP_001029372.1): | |
| 1 | mqvstaalav llctvalcnr isatfaadtp tsccfsyisr qipqnfiady |
| | fetnsqcskp |
| 61 | gvifltkrgr qvcadpskew vqkyvsdlel sa |
| Human CCL4 coding nucleic acid sequence (NCBI Accession NM_002984.2): | |
| 1 | atgaagctct gcgtgactgt cctgtctctc ctcatgctag tagctgcctt |
| | ctgctctcca |
| 61 | gcgctctcag caccaatggg ctcagaccct cccaccgcct gctgcttttc |
| | ttacactgcg |
| 121 | aggaagcttc ctcgcaactt tgtggtagat tactatgaga ccagcagcct |
| | ctgctcccag |
| 181 | ccagctgtgg tattccaaac caaaagaagc aagcaagtct gtgctgatcc |
| | cagtgaatcc |
| 241 | tgggtccagg agtacgtgta tgacctggaa ctgaactga |
| Human CCL4 protein sequence (NCBI Accession NP_002975.1): | |
| 1 | mklcvtvls1 lmlvaafcsp alsapmgsdp ptaccfsyta rklprnfvvd |
| | yyetsslcsq |
| 61 | pavvfqtkrs kqvcadpses wvqeyvydle ln |
| Monkey CCL4 coding nucleic acid sequence (NCBI Accession NM_001032873.1): | |
| 1 | atgaagctct gcgtgactgt cctgtctctc ctcgtgctag cagctgcctt |
| | ctgctctcca |
| 61 | gcactctcag caccaatggg ctcagaccct cccacctcct gctgcttttc |
| | ttacaccgcg |
| 121 | aggaagcttc ctcgcaactt tgtggtagat tactacgaga ccagcagcct |
| | ctgctcccag |
| 181 | ccagctgtgg tattccagac caaaagaggc aagcaagtct gcgctgaccc |
| | cagtgagacc |
| 241 | tgggtccagg agtatgttaa tgacctggaa ctgaactga |
| Monkey CCL4 protein sequence (NCBI Accession NP_001028045.1): | |
| 1 | mklcvtvls1 lvlaaafcsp alsapmgsdp ptsccfsyta rklprnfvvd |
| | yyetsslcsq |
| 61 | pavvfqtkrg kqvcadpset wvqeyvndle ln |
| Human CCL5 coding nucleic acid sequence (NCBI Accession NM_002985.2): | |
| 1 | atgaaggtct ccgcggcagc cctcgctgtc atcctcattg ctactgccct |
| | ctgcgctcct |
| 61 | gcatctgcct ccccatattc ctcggacacc acaccctgct gctttgccta |
| | cattgcccgc |
| 121 | ccactgcccc gtgcccacat caaggagtat ttctacacca gtggcaagtg |
| | ctccaaccca |
| 181 | gcagtcgtct ttgtcacccg aaagaaccgc caagtgtgtg ccaacccaga |
| | gaagaaatgg |
| 241 | gttcgggagt acatcaactc tttggagatg agctag |
| Human CCL5 protein sequence (NCBI Accession NP_002976.2): | |
| 1 | mkvsaaalav iliatalcap asaspyssdt tpccfayiar plprahikey |
| | fytsgkcsnp |
| 61 | avvfvtrknr qvcanpekkw vreyinslem s |
| Monkey CCL5 coding nucleic acid sequence (NCBI Accession NM_001032850.1): | |
| 1 | atgaaggtct ccgcggcacg cctcgctgtc atcctcgttg ctacagccct |
| | ctgcgctcct |
| 61 | gcatctgcct ccccacatgc ctcagacacc acaccctgct gctttgccta |
| | cattgcccgc |
| 121 | ccactgcccc gtgcccacat caaggagtat ttctacacca gtggcaagtg |
| | ctccaaccca |
| 181 | gcagtcgtct ttgtcacccg aaagaatcgc caagtgtgtg ccaacccaga |
| | gaagaaatgg |
| 241 | gttcgggagt acatcaactc tttggagatg agctag |
| Monkey CCLS protein sequence (NCBI Accession NP_001028022.1): | |
| 1 | mkvsaarlav ilvatalcap asasphasdt tpccfayiar plprahikey |
| | fytsgkcsnp |
| 61 | avvfvtrknr qvcanpekkw vreyinslem s |
| Human CCR5 coding nucleic acid sequence (NCBI Accession NM_002985.2 and | |
| NM_001100168.1): | |
| 1 | atggattatc aagtgtcaag tccaatctat gacatcaatt attatacatc |
| | ggagccctgc |
| 61 | caaaaaatca atgtgaagca aatcgcagcc cgcctcctgc ctccgctcta |
| | ctcactggtg |
| 121 | ttcatctttg gttttgtggg caacatgctg gtcatcctca tcctgataaa |
| | ctgcaaaagg |
| 181 | ctgaagagca tgactgacat ctacctgctc aacctggcca tctctgacct |
| | gtttttcctt |
| 241 | cttactgtcc ccttctgggc tcactatgct gccgcccagt gggactttgg |
| | aaatacaatg |
| 301 | tgtcaactct tgacagggct ctattttata ggcttcttct ctggaatctt |
| | cttcatcatc |
| 361 | ctcctgacaa tcgataggta cctggctgtc gtccatgctg tgtttgcttt |
| | aaaagccagg |
| 421 | acggtcacct ttggggtggt gacaagtgtg atcacttggg tggtggctgt |
| | gtttgcgtct |
| 481 | ctcccaggaa tcatctttac cagatctcaa aaagaaggtc ttcattacac |
| | ctgcagctct |
| 541 | cattttccat acagtcagta tcaattctgg aagaatttcc agacattaaa |
| | gatagtcatc |
| 601 | ttggggctgg tcctgccgct gcttgtcatg gtcatctgct actcgggaat |
| | cctaaaaact |
| 661 | ctgcttcggt gtcgaaatga gaagaagagg cacagggctg tgaggcttat |
| | cttcaccatc |
| 721 | atgattgttt attttctctt ctgggctccc tacaacattg tccttctcct |
| | gaacaccttc |
| 781 | caggaattct ttggcctgaa taattgcagt agctctaaca ggttggacca |
| | agctatgcag |
| 841 | gtgacagaga ctcttgggat gacgcactgc tgcatcaacc ccatcatcta |
| | tgcctttgtc |
| 901 | ggggagaagt tcagaaacta cctcttagtc ttcttccaaa agcacattgc |
| | caaacgcttc |
| 961 | tgcaaatgct gttctatttt ccagcaagag gctcccgagc gagcaagctc |
| | agtttacacc |
| 1021 | cgatccactg gggagcagga aatatctgtg ggcttgtga |
| Human CCR5 protein sequence (NCBI Accession NP_000570.1 and NP_001093638.1): | |
| 1 | mdyqvsspiy dinyytsepc qkinvkqiaa rllpplyslv fifgfvgnml |
| | vililinckr |
| 61 | lksmtdiyll nlaisdlffl ltvpfwahya aaqwdfgntm cqlltglyfi |
| | gffsgiffii |
| 121 | lltidrylav vhavfalkar tvtfgvvtsv itwvvavfas lpgiiftrsq |
| | keglhytcss |
| 181 | hfpysqyqfw knfqtlkivi lglvlpllvm vicysgilkt llrcrnekkr |
| | hravrlifti |
| 241 | mivyflfwap ynivlllntf qeffglnncs ssnrldqamq vtetlgmthc |
| | cinpiiyafv |
| 301 | gekfrnyllv ffqkhiakrf ckccsifqqe aperassvyt rstgeqeisv gl |
| Monkey CCR5 coding nucleic acid sequence (NCBI Accession NM_001042773.2): | |
| 1 | atggactatc aagtgtcaag tccaacctat gacatcgatt attatacatc |
| | ggaaccctgc |
| 61 | caaaaaatca atgtgaaaca aatcgcagcc cgcctcctgc ctccgctcta |
| | ctcactggtg |
| 121 | ttcatctttg gttttgtggg caacatactg gtcgtcctca tcctgataaa |
| | ctgcaaaagg |
| 181 | ctgaaaagca tgactgacat ctacctgctc aacctggcca tctctgacct |
| | gcttttcctt |
| 241 | cttactgtcc ccttctgggc tcactatgct gctgcccagt gggactttgg |
| | aaatacaatg |
| 301 | tgtcaactct tgacagggct ctattttata ggcttcttct ctggaatctt |
| | cttcatcatc |
| 361 | ctcctgacaa tcgataggta cctggctatc gtccatgctg tgtttgcttt |
| | aaaagccagg |
| 421 | acagtcacct ttggggtggt gacaagtgtg atcacttggg tggtggctgt |
| | gtttgcctct |
| 481 | ctcccaggaa tcatctttac cagatctcag agagaaggtc ttcattacac |
| | ctgcagctct |
| 541 | cattttccat acagtcagta tcaattctgg aagaattttc agacattaaa |
| | gatggtcatc |
| 601 | ttggggctgg tcctgccgct gcttgtcatg gtcatctgct actcgggaat |
| | cctgaaaact |
| 661 | ctgcttcggt gtcgaaacga gaagaagagg cacagggctg tgaggcttat |
| | cttcaccatc |
| 721 | atgattgttt attttctctt ctgggctccc tacaacattg tccttctcct |
| | gaacaccttc |
| 781 | caggaattct ttggcctgaa taattgcagt agctctaaca ggttggacca |
| | agccatgcag |
| 841 | gtgacagaga ctcttgggat gacacactgc tgcatcaacc ccatcatcta |
| | tgccttygtc |
| 901 | ggggagaagt tcagaaacta cctcttagtc ttcttccaaa agcacattgc |
| | caaacgcttc |
| 961 | tgcaaatgct gttccatttt ccagcaagag gctcccgagc gagcaagttc |
| | agtttacacc |
| 1021 | cgatccactg gggagcagga aatatctgtg ggcttgtga |
| Monkey CCR5 protein sequence (NCBI Accession NP_001036238.2): | |
| 1 | mdyqvsspty didyytsepc qkinvkqiaa rllpplyslv fifgfvgnil |
| | vvlilinckr |
| 61 | lksmtdiyll nlaisdllfl ltvpfwahya aaqwdfgntm cqlltglyfi |
| | gffsgiffii |
| 121 | lltidrylai vhavfalkar tvtfgvvtsv itwvvavfas lpgiiftrsq |
| | reglhytcss |
| 181 | hfpysqyqfw knfqtlkmvi lglvlpllvm vicysgilkt llrcrnekkr |
| | hravrlifti |
| 241 | mivyflfwap ynivlllntf qeffglnncs ssnrldqamq vtetlgmthc |
| | cinpiiyafv |
| 301 | gekfrnyllv ffqkhiakrf ckccsifqqe aperassvyt rstgeqeisv gl |

The present invention further provides variants, fragments, and functionally similar homologs of chemokine ligands and chemokine receptors for use in the methods described herein. For example, a nucleic acid molecule of the present invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a protein which corresponds to a marker of the invention, and thus encode the same protein.

In addition to the nucleotide sequences described in the Tables, Figures, and Sequence Listing described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene described herein can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989).

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of one of the proteins which correspond to the markers of the invention.

### III. METHODS OF THE INVENTION

The present invention provides methods to prevent or treat cardiac and neurological disorders, as well as to increase responsiveness of a subject having such a disorder to a treatment regimen by inhibiting or reducing the amount and/or activity of a chemokine receptor in cardiac (e.g., cardiomyocytes) or neural cells (e.g., CNS or PNS neurons). A method may include reducing the expression of a chemokine receptor and/or chemokine ligand gene, reducing the amount of chemokine receptor and/or chemokine ligand protein, or inhibiting the activity of a chemokine receptor and/or chemokine ligand protein.

Prophylaxis may be appropriate even at very early stages of the disease, to prevent recurrent tumorigenesis or metastasis. Thus, administration of an agent that reduces chemokine receptor and/or chemokine ligand levels or activity may be effected as soon as a PIV infection, cardiac disorder, and/or neurological disorder is diagnosed, and treatment continued for as long as is necessary, generally until the threat of the infection or disorder has been removed. Such treatment may also be used prophylactically.

1. RNAi Technology

In one embodiment, chemokine receptor amount and/or activity is antagonized by administration of or expression in a subject, e.g., in cells or a tissue of the subject, of one or more siRNAs.

Isolated RNA molecules specific to chemokine receptor and/or chemokine ligand mRNA, which mediate RNAi, are antagonists useful in the method of the present invention (see, e.g., U.S. Patent Application Nos: 20030153519A1; 20030167490A1; and U.S. Pat. Nos. 6,506,559; 6,573,099, which are herein incorporated by reference in their entirety).

In one embodiment, the RNA is comprised of, or capable of being cleaved to, short interfering or small interfering RNAs (siRNAs). The term “short interfering RNAs (siRNA)” as used herein is intended to refer to any nucleic acid molecule capable of mediating RNAi or gene silencing. The term siRNA is intended to encompass various naturally generated or synthetic compounds, with RNAi function. Such compounds include, without limitation, duplex synthetic oligonucleotides, of about 21 to 23 base pairs with terminal overlaps of 2 or 3 base pairs; hairpin structures of one oligonucleotide chain with sense and complementary, hybridizing, segments of 21-23 base pairs joined by a loop of 3-5 base pairs; and various genetic constructs leading to the expression of the preceding structures or functional equivalents. Such genetic constructs are usually prepared in vitro and introduced in the test system, but can also include siRNA from naturally occurring siRNA precursors coded by the genome of the host cell or animal.

It is not a requirement that the siRNA be comprised solely of RNA. In one embodiment, the siRNA comprises one or more chemical modifications and/or nucleotide analogues. The modification and/or analogue may be any modification and/or analogue, respectively, that does not negatively affect the ability of the siRNA to inhibit chemokine ligand and/or chemokine receptor expression. The inclusion of one or more chemical modifications and/or nucleotide analogues in an siRNA may be used to prevent or slow nuclease digestion, and in turn, create a more stable siRNA for practical use. Chemical modifications and/or nucleotide analogues which stabilize RNA are known in the art. Phosphorothioate derivatives, which include the replacement of non-bridging phosphoryl oxygen atoms with sulfur atoms, are one example of analogues showing increased resistance to nuclease digestion. Sites of the siRNA which may be targeted for chemical modification include the loop region of a hairpin structure, the 5′ and 3′ ends of a hairpin structure (e.g. cap structures), the 3′ overhang regions of a double-stranded linear siRNA, the 5′ or 3′ ends of the sense strand and/or antisense strand of a linear siRNA, and one or more nucleotides of the sense and/or antisense strand.

As used herein, the term siRNA is intended to be equivalent to any term in the art defined as a molecule capable of mediating sequence-specific RNAi. Such equivalents include, for example, double-stranded RNA (dsRNA), microRNA (mRNA), short hairpin RNA (shRNA), short interfering oligonucleotide, and post-transcriptional gene silencing RNA (ptgsRNA).

siRNAs may be introduced into cells to suppress gene expression for therapeutic or prophylactic purposes as described in International Publication Number WO 0175164. Such molecules may be introduced into cells to suppress gene expression for therapeutic or prophylactic purposes as described in various patents, patent applications and papers. Publications herein incorporated by reference, describing RNAi technology include, but are not limited to, the following: U.S. Pat. No. 6,686,463, U.S. Pat. No. 6,673,611, U.S. Pat. No. 6,623,962, U.S. Pat. No. 6,506,559, U.S. Pat. No. 6,573,099, and U.S. Pat. No. 6,531,644; International Publication Numbers WO04061081; WO04052093; WO04048596; WO04048594; WO04048581; WO04048566; WO04046320; WO04044537; WO04043406; WO04033620; WO04030660; WO04028471; WO 0175164. Papers which describe the methods and concepts for the optimal use of these compounds include, but are not limited to, the following: Brummelkamp, Science 296: 550-553 (2002); Caplen, Expert Opin. Biol. Ther. 3:575-86 (2003); Brummelkamp, Science Express 21 March 3 1-6 (2003); Yu, Proc Natl Acad Sci USA 99:6047-52 (2002); Paul, Nature Biotechnology 29:505-8 (2002); Paddison, Proc Natl Acad Sci USA 99:1443-8 (2002); Brummelkamp, Nature 424: 797-801 (2003); Brummelkamp, Science 296:550-3 (2003); Sui, Proc Natl Acad Sci USA 99: 5515-20 (2002); Paddison, Genes and Development 16:948-58 (2002).

A composition comprising an siRNA effective to inhibit chemokine receptor and/or chemokine ligand expression may include an RNA duplex comprising a portion of the sense sequence of the chemokine receptor and/or chemokine ligand. In this embodiment, the RNA duplex comprises a first strand comprising a sense sequence of the chemokine receptor and/or chemokine ligand and a second strand comprising a reverse complement of the sense sequence of the chemokine receptor and/or chemokine ligand. In one embodiment the sense sequence of the chemokine receptor and/or chemokine ligand comprises of from 10 to 25 nucleotides in length. In another embodiment, the sense sequence of the chemokine receptor and/or chemokine ligand comprises of from 19 to 25 nucleotides in length. In yet another embodiment, the sense sequence of the chemokine receptor and/or chemokine ligand comprises of from 21 to 23 nucleotides in length. The sense sequence of the chemokine receptor and/or chemokine ligand can comprises a sequence of the chemokine receptor and/or chemokine ligand containing a translational start site or a portion of the chemokine receptor and/or chemokine ligand sequence within the first 400 nucleotides of the chemokine receptor and/or chemokine ligand mRNA.

In another embodiment, a composition comprising an siRNA effective to inhibit chemokine receptor and/or chemokine ligand expression may comprise in a single molecule a sense sequence of the chemokine receptor and/or chemokine ligand, the reverse complement of the sense sequence of the chemokine receptor and/or chemokine ligand, and an intervening sequence enabling duplex formation between the sense and reverse complement sequences. The sense sequence of the chemokine receptor and/or chemokine ligand may comprise 10 to 25 nucleotides in length, 19 to 25 nucleotides in length, or 21 to 23 nucleotides in length.

It will be readily apparent to one of skill in the art that an siRNA of the present invention may comprise a sense sequence of the chemokine receptor and/or chemokine ligand or the reverse complement of the sense sequence of the chemokine receptor and/or chemokine ligand, which is less than perfectly complementary to each other or to the targeted region of the chemokine receptor and/or chemokine ligand. In other words, the siRNA may comprise mismatches or bulges within the sense or reverse complement sequence. In one aspect, the sense sequence or its reverse complement may not be entirely contiguous. The sequence or sequences may comprise one or more substitutions, deletions, and/or insertions. The only requirement of the present invention is that the siRNA sense sequence possess enough complementarity to its reverse complement and to the targeted region of the chemokine receptor and/or chemokine ligand to allow for RNAi activity. It is an object of the present invention, therefore, to provide for sequence modifications of an siRNA of the present invention that retain sufficient complementarity to allow for RNAi activity. One of skill in the art may predict that a modified siRNA composition of the present invention will work based on the calculated binding free energy of the modified sequence for the complement sequence and targeted region of the chemokine receptor and/or chemokine ligand. Methods for calculating binding free energies for nucleic acids and the effect of such values on strand hybridization are known in the art.

A wide variety of delivery systems are available for use in delivering an siRNA of the present invention to a target cell in vitro and in vivo. An siRNA of the present invention may be introduced directly or indirectly into a cell in which chemokine receptor and/or chemokine ligand inhibition is desired. An siRNA may be directly introduced into a cell by, for example, injection. As such, it is an object of the invention to provide for a composition comprising an siRNA effective to inhibit the chemokine receptor and/or chemokine ligand in injectable, dosage unit form. An siRNA of the present invention may be injected intravenously or subcutaneously, as an example, for therapeutic use in conjunction with the methods and compositions of the present invention. Such treatment may include intermittent or continuous administration until therapeutically effective levels are achieved to inhibit chemokine receptor and/or chemokine ligand expression in the desired tissue.

Indirectly, an expressible DNA sequence or sequences encoding the siRNA may be introduced into a cell and the siRNA, thereafter, transcribed from the DNA sequence or sequences. It is an object of the present invention, therefore, to provide for compositions comprising a DNA sequence or sequences which encode an siRNA effective to inhibit chemokine receptor and/or chemokine ligand expression.

A DNA composition of the present invention comprises a first DNA sequence which encodes a first RNA sequence comprising a sense sequence of the chemokine receptor and/or chemokine ligand and a second DNA sequence which encodes a second RNA sequence comprising the reverse complement of the sense sequence of the chemokine receptor and/or chemokine ligand. The first and second RNA sequences, when hybridized, form an siRNA duplex capable of forming an RNA-induced silencing complex, the RNA-induced silencing complex being capable of inhibiting chemokine receptor and/or chemokine ligand expression. The first and second DNA sequences may be chemically synthesized or synthesized by PCR using appropriate primers to the chemokine receptor and/or chemokine ligand. Alternatively, the DNA sequences may be obtained by recombinant manipulation using cloning technology, which is well known in the art. Once obtained, the DNA sequences may be purified, combined, and then introduced into a cell in which chemokine receptor and/or chemokine ligand inhibition is desired. Alternatively, the sequences may be contained in a single vector or separate vectors and the vector or vectors introduced into the cell in which chemokine receptor and/or chemokine ligand inhibition is desired.

Delivery systems available for use in delivering a DNA composition of the present invention to a target cell are described further below and include, for example, viral and non-viral systems. Examples of suitable viral systems include, for example, adenoviral vectors, adeno-associated virus, lentivirus, poxvirus, retroviral vectors, vaccinia, herpes simplex virus, HIV, the minute virus of mice, hepatitis B virus and influenza virus. Non-viral delivery systems may also be used, for example using, uncomplexed DNA, DNA-liposome complexes, DNA-protein complexes and DNA-coated gold particles, bacterial vectors such as salmonella, and other technologies such as those involving VP22 transport protein, Co-X-gene, and replicon vectors. A viral or non-viral vector in the context of the present invention may express the antigen of interest.

2. Antisense Technology

In another embodiment, the level of chemokine receptor and/or chemokine ligand is reduced or decreased by administration or the expression of antisense molecules in a subject or tissue or cell thereof. An antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

The antisense nucleic acids and oligonucleotides of the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids. For example, phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1 (1)1986.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

3. Other Nucleic Acid-Based Chemokine Receptor Antagonists

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

In addition, aptamers having antibody binding-like properties are well known in the art and can be produced using the methodology disclosed in a U.S. Pat. No. 5,270,163 and WO 91/19813.

4. Chemokine Receptor Antagonist Polypeptides

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Such variants are useful for generating polypeptides having chemokine receptor antagonist properties. For example, a polypeptide variant of a chemokine ligand may maintain the ability to bind a chemokine receptor without activating the receptor or a soluble fragment of a chemokine receptor binding domain may bind to and sequester available chemokine ligands to thereby antagonize chemokine receptor signaling. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Polypeptides, as well as fragments or variants thereof, can further be made and used. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants such as dominant-negative variants (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes the chemokine ligand or chemokine receptor. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

In one embodiment, the peptide has an amino acid sequence identical or similar to a chemokine ligand, chemokine receptor, or chemokine receptor antagonist. In one embodiment, the peptide competes with a chemokine ligand polypeptide or a fragment thereof for binding its natural binding partner(s) chemokine receptor, or a fragment(s) thereof.

Peptides can be produced, typically by direct chemical synthesis, and used e.g., as antagonists of the interactions between a chemokine ligand, or a fragment thereof, and its natural binding partner(s) chemokine receptor, or a fragment(s) thereof. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, and biochemical properties.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to peptides useful for diagnostic, prognostic, and/or clinical trial monitoring applications can be used to produce equivalent diagnostic, prognostic, and/or clinical trial monitoring applications. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds. Derivitization (e.g., labeling) of peptidomimetics should not substantially interfere with the desired diagnostic and/or prognostic utility of the peptidomimetic.

These peptides or peptidomimetic molecules can also be chimeric or fusion proteins. As used herein, a “chimeric protein” or “fusion protein” comprises a protein, peptide, or peptidomimetic molecule or a fragment thereof operatively linked to another protein, peptide, or peptidomimetic molecule or a fragment thereof. Within the chimeric or fusion protein, the term “operatively linked” is intended to indicate that the independent protein, peptide, or peptidomimetic molecules or fragments thereof are fused in-frame to each other in such a way as to preserve functions exhibited when expressed independently of the fusion.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. Preferably, the first peptide consists of a portion of a chemokine ligand, chemokine receptor, or chemokine receptor antagonist that comprises at least one biologically active portion of the chemokine ligand, chemokine receptor, or chemokine receptor antagonist, respectively. In another preferred embodiment, the first peptide consists of a portion of a biologically active molecule. The second peptide can include an immunoglobulin constant region, for example, a human Cγ1 domain or Cγ₄ domain (e.g., the hinge, CH2 and CH3 regions of human IgCγ1, or human IgCγ4, see e.g., Capon et al. U.S. Pat. No. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). A polypeptide encoding nucleic acid can be cloned into such an expression vector such that the fusion moiety is linked in-frame to the chemokine ligand- or chemokine receptor-encoding sequences.

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between a chemokine ligand, or a fragment thereof, and its natural binding partner(s) chemokine receptor, or a fragment(s) thereof.

5. Anti-Chemokine Ligand and/or Anti-Chemokine Receptor Antibodies

In yet another embodiment, chemokine receptor amount and/or activity is reduced by administration to or expression in a subject or a cell or tissue thereof, of anti-chemokine ligand and/or anti-chemokine receptor antibodies. Accordingly, anti-chemokine ligand and/or anti-chemokine receptor antibodies can be used_as chemokine receptor antagonists according to the methods of the present invention. Many such antibodies are known in the art. Alternatively, an isolated chemokine ligand or chemokine receptor polypeptide or a fragment thereof (or a nucleic acid encoding such a polypeptide), can be used as an immunogen to generate antibodies that bind to said immunogen, using standard techniques for polyclonal and monoclonal antibody preparation. A full-length polypeptide can be used, or alternatively, the disclosure relates to antigenic peptide fragments of the polypeptide for use as immunogens. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody binds substantially specifically to a chemokine ligand or chemokine receptor, or a fragment thereof. In a preferred embodiment, an antibody binds to a CCL3, CCL4, CCL5, or CCR5 polypeptide, or a fragment thereof, and blocks the interaction between the chemokine ligand, or a fragment thereof, and its natural binding partner(s) chemokine receptor, or a fragment(s) thereof.

An immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating a monoclonal antibody (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal antibody specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. No. 5,565,332, 5,871,907, or 5,733,743.

Additionally, fully human antibodies could be made against the immunogen. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences.

6. Small Molecule Chemokine Receptor Antagonists

Compounds that inhibit the activity of chemokine receptors may also be used. Such compounds include small molecules, e.g., molecules that interact with the active site or a binding site of the protein, e.g., an RNA binding site. Such compounds are well known in the art and additional compounds may be identified according to methods known in the art. For example, small molecule antagonists of the CCR5 chemokine receptor include, but are not limited to, maraviroc, vicrviroc, NCB-9471, PRO-140, CCR5 mAb004, 8-[4-(2-butoxyethoxy)phenyl]-1-isobutyl-N-[4-[[(1-propyl-1H-imadazol-5-yl-)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicy-clo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carbox-ylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carb-oxylate, and N-{(1S)-3-[3-endo-(5-isobutyryl-2-methyl-4,5,6,7-tetrahydro-1H-imidazo[4,-5-c]pyridin-yl)-8-azabicyclo[3.2.1]oct-8-yl]-1-(3-fluorophenyl)propyl}acet-amide), Sch-C, Sch-D, TAK-220, PRO-140, and/or a pharmaceutically acceptable salt and/or solvate thereof.

7. Combination Compositions

In addition to pharmaceutically active carriers, diluent, solvent, vehicles, and the like, one or more agents may further be combined with a chemokine receptor antagonist in order to further treat a subject having a condition in need thereof.

For subjects infected with a PIV, antiviral agents can be co-administered. For example, protease inhibitors (PI), integrase inhibitors (II), and/or nucleoside/nucleotide reverse transcriptase inhibitors (NRTI) can be co-administered. Examples of PIs include, but are not limited to, amprenavir (141W94), CGP-73547, CGP-61755, DMP-450 (mozenavir), nelfinavir, ritonavir, saquinavir, lopinavir, TMC-126, atazanavir, palinavir, GS-3333, KN 1-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium, benzenesulfonamide derivatives disclosed in WO 03/053435, R-944, Ro-03-34649, VX-385, GS-224338, OPT-TL3, PL-100, PPL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005. Examples of IIs include, but are not limited to, L-000870810 GW-810781, 1,5-naphthyridine-3-carboxamide derivatives, 5-hydroxypyrimidine-4-carboxamide derivatives, MK-0518, and GS-9137 (JTK-303), (5-(1,1-dioxo-1,2-thiazinan-2-yl)-N-(4-fluorobenzyl)-8-hydroxy-1,6-naphth-yridine-7-carboxamide), and GSK-364735. Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC).

It may further be advantageous to administer the regimens described herein with other agents, such as proteins, peptides, antibodies, and the like to treat cardiac-related disorders and/or ameliorate their symptoms. For example, inhibitors of the rennin-angiotensin system and/or β-adrenergic blocking agents can be used. Therapeutic agents to treat pathologic hypertrophy in the setting of heart failure include angiotensin II converting enzyme (ACE) inhibitors and β-adrenergic receptor blocking agents. Non-pharmacological treatment is primarily used as an adjunct to pharmacological treatment. One means of non-pharmacological treatment involves reducing the sodium in the diet. In addition, non-pharmacological treatment can include the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

Non-limiting examples of a pharmacological therapeutic agent that may be used in the present invention include an antihyperlipoproteinemic agent (e.g., an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof), an antiarteriosclerotic agent (e.g., pyridinol carbamate), an antithrombotic/fibrinolytic agent (e.g., anticoagulants, anticoagulant antagonists, antiplatelet agents, thrombolytic agents, thrombolytic agent antagonists or combinations thereof), a blood coagulant (e.g., thrombolytic agent antagonists and anticoagulant antagonists), an antiarrhythmic agent (e.g., Class I antiarrythmic agents (sodium channel blockers), Class II antiarrythmic agents (β-adrenergic blockers), Class II antiarrythmic agents (repolarization prolonging drugs), Class IV antiarrhythmic agents (calcium channel blockers) and miscellaneous antiarrythmic agents), an antihypertensive agent (e.g., sympatholytic, alpha/beta blockers, alpha blockers, anti-angiotensin II agents, beta blockers, calcium channel blockers, vasodilators and miscellaneous antihypertensives), a vasopressor (e.g., amezinium methyl sulfate, angiotensin amide, dimetofrine, dopamine, etifelmin, etilefrin, gepefrine, metaraminol, midodrine, norepinephrine, pholedrine and synephrine), a treatment agent for congestive heart failure (e.g., anti-angiotensin II agents, afterload-preload reduction treatment, diuretics and inotropic agents, an antianginal agent, an antibacterial agent or a combination thereof.

Non-limiting examples of a surgical therapeutic agent that may be used in the present invention include preventive, diagnostic or staging, curative, and palliative surgery. Surgery, and in particular, a curative surgery, may be used in conjunction with other therapies, such as the present invention and one or more other agents. Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

8. Pharmaceutical Formulations

The present invention provides pharmaceutically acceptable compositions which comprise a therapeutically-effective amount of a composition described herein, formulated together with one or more pharmaceutically acceptable carriers (additives) and/or diluents and with or without additional antiviral agents and/or immunogens. As described in detail below, the pharmaceutical compositions of the present invention can be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

It will be appreciated that individual dosages can be varied depending upon the requirements of the subject in the judgment of the attending clinician, the severity of the condition being treated and the particular compound being employed. In determining the therapeutically effective amount or dose, a number of additional factors can be considered by the attending clinician, including, but not limited to: the pharmacodynamic characteristics of the particular agent and its mode and route of administration; the desired time course of treatment; the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the kind of concurrent treatment; and other relevant circumstances.

Treatment can be initiated with smaller dosages which are less than the effective dose of the compound. Thereafter, in one embodiment, the dosage should be increased by small increments until the optimum effect under the circumstances is reached. For convenience, the total daily dosage can be divided and administered in portions during the day if desired.

The duration and/or dose of treatment with therapies can vary according to the particular agent or combination thereof. An appropriate treatment time for a particular therapeutic agent will be appreciated by the skilled artisan. The invention contemplates the continued assessment of optimal treatment schedules for each therapeutic agent, where the phenotype of the cardiac-related disorder of the subject as determined by the methods of the invention is a factor in determining optimal treatment doses and schedules.

In general, it is preferable to obtain a first sample from the subject prior to begining therapy and one or more samples during treatment. In such a use, a baseline of expression of cells from subjects with a cardiac-related disorder prior to therapy is determined and then changes in the baseline state of expression of cells from subjects with a cardiac-related disorder is monitored during the course of therapy. Alternatively, two or more successive samples obtained during treatment can be used without the need of a pre-treatment baseline sample. In such a use, the first sample obtained from the subject is used as a baseline for determining whether the expression of cells from subjects with a cardiac-related disorder is increasing or decreasing.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) chemokine ligand and/or chemokine receptor expression and/or activity, or expression and/or activity of the complex encompassed by the invention. These salts can be prepared in situ during the final isolation and purification of the agents, or by separately reacting a purified agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates ((e.g., inhibits) chemokine ligand and/or chemokine receptor expression and/or activity, or expression and/or activity of the complex. These salts can likewise be prepared in situ during the final isolation and purification of the agents, or by separately reacting the purified agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) chemokine ligand and/or chemokine receptor expression and/or activity, with the carrier and, optionally, one or more accessory ingredients. In general, the formulations are prepared by uniformly and intimately bringing into association an agent with liquid carriers, or finely divided solid carriers, or both, and then, if necessary, shaping the product.

Formulations suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. A compound can also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered peptide or peptidomimetic moistened with an inert liquid diluent.

Tablets, and other solid dosage forms, such as dragees, capsules, pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration can be presented as a suppository, which can be prepared by mixing one or more agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) chemokine ligand and/or chemokine receptor expression and/or activity include powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to an agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) chemokine ligand and/or chemokine receptor expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) chemokine ligand and/or chemokine receptor expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of an agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) chemokine ligand and/or chemokine receptor expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

Actual dosage levels of the active ingredients in the pharmaceutical compositions of this invention can be determined by the methods of the present invention so as to obtain an amount of the active ingredient, which is effective to achieve the desired therapeutic response for a particular subject, composition, and mode of administration, without being toxic to the subject.

Nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Formulations may be any that are appropriate to the route of administration, and will be apparent to those skilled in the art (e.g., by injection, infusion, aerosol, oral, transdermal, transmucosal, intrapleural, intrathecal, or other suitable routes). Preferably, the composition is administered by intravenous, subcutaneous, intradermal, or intramuscular routes, or any combination thereof.

Representative forms of administrable nucleic acids include a “naked” DNA plasmid, a “naked” RNA molecule, a DNA molecule packaged into a replicating or nonreplicating viral vector, an RNA molecule packaged into a replicating or nonreplicating viral vector, a DNA molecule packaged into a bacterial vector, or combinations thereof.

In one embodiment, recombinant chemokine ligand and/or chemokine receptor polypeptides can be administered to subjects. In some embodiments, fusion proteins can be constructed and administered which have enhanced biological properties. In addition, the polypeptides can be modified according to well-known pharmacological methods in the art (e.g., pegylation, glycosylation, oligomerization, etc.) in order to further enhance desirable biological activities, such as increased immunogenicity, bioavailability, and/or decreased proteolytic degradation.

For embodiments using instant nucleic acid delivery, any means for the introduction of a polynucleotide into a subject, such as a human or non-human mammal, or cells thereof can be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs can first be experimentally optimized for expression (e.g., inclusion of an intron in the 5′ untranslated region and elimination of unnecessary sequences (Feigner, et al., Ann NY Acad Sci 126-139, 1995). Formulation of DNA, e.g. with various lipid or liposome materials, can then be effected using known methods and materials and delivered to the recipient mammal. See, e.g., Canonico et al., Am J Respir Cell Mol Biot 10:24-29, 1994; Tsan et al., Am J Physiol 268; Alton et al., Nat. Genet. 5:135-142, 1993; and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system can be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest can be in a plasmid or viral vector, or other vector as is known in the art. Such vectors are well known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter can be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences, such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the invention have been derived from herpes virus, e.g., Herpes Simplex Virus (U.S. Pat. No. 5,631,236 by Woo et al., issued Can 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxvirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

For some methods, local administration into cardiac tissue may be desired and methods are known in the art for achieving such local administration. For example, a composition can be injected into an affected vessel (e.g., an artery) or an organ (e.g., the heart). In one method of treatment embodiment, flow of blood through coronary vessels of a heart is restricted and a viral delivery system is introduced into the lumen of a coronary artery. In one embodiment, the heart is permitted to pump while coronary vein outflow is restricted. In another embodiment, a viral delivery system is used and injected into the heart while restricting aortic flow of blood out of the heart, thereby allowing the viral delivery system to flow in to and be delivered to the heart. In other embodiments, the flow of blood through the coronary vessels is completely restricted, and in specific such embodiments, the restricted coronary vessels comprise: the left anterior descending artery (LAD, the distal circumflex artery (LCX), the great coronary vein (GCV), the middle cardiac vein (MCV), or the anterior interventricular vein (AIV). In certain embodiments, the introduction of the viral delivery system occurs after ischemic preconditioning of the coronary vessels. In another embodiment, the composition is injected into the heart by a method comprising the steps of restricting aortic flow of blood out of the heart, such that blood flow is re-directed to coronary arteries; injecting the vector into lumen of the heart, aorta or coronary ostia such that the vector flows into the coronary arteries; permitting the heart to pump while the aortic flow of blood out of the heart is restricted; and reestablishing the aortic flow of blood. In still another embodiment, a composition is injected into the heart with a catheter. In yet another embodiment, a composition is directly injected into a muscle of the heart.

For other methods, particular methods for administration to CNS or PNS cells may be appropriate and are well known to the skilled artisan. Subcutaneous administration of an active agent can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system. Such delivery methods may be especially useful for administration to cell types in the CNS.

In some embodiments, an active agent (e.g., a cyclophilin inhibitor, at least one additional therapeutic agent, etc.) is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art. Such delivery methods may be especially useful for administration to cell types in the CNS.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned, such as a site within the CNS. Implantation sites include, but are not necessarily limited to a subdermal, subcutaneous, intramuscular, or other suitable site within a subject's body. Subcutaneous implantation sites are generally used because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump are also suitable for use with the present invention. Examples of such devices include those described in, for example, U.S. Pat. Nos. 4,692,147; 4,360,019; 4,487,603; 4,360,019; 4,725,852, and the like. In general, a subject treatment method can be carried out using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in a subject treatment method include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted above, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

9. Methods of Evaluating Cardiac-Related Responses

In some aspects, the present invention requires the analysis of cardiac-related responses. Without limitation, a representative list of cardiac-related responses to be analyzed include one or more of a) decreased cardiomyocyte contractility; b) decreased left ventricular ejection fraction and/or volume; c) increased end-systolic and/or end-diastolic fractional shortening; d) decreased mitral valve annular velocity; e) decreased mitral inflow; f) decreased aortic velocity time integral; g) decreased aorta cross section area; h) decreased isovolumetric contraction time; i) increased isovolumetric relaxation time; j) decreased heart chamber mechanical efficiency; k) increased cardiac protein kinase A and/or protein kinase C biomarker expression or activity; 1) decreased phosphorylation of cardiac myofilament protein biomarker; m) increased macrophage activation and/or infiltration in the myocardium; n) increased chemokine biomarker expression and/or activity in the myocardium; o) increased cytokine biomarker expression and/or activity in the myocardium; p) increased cardiac fibrosis; q) increased cardiac inflammation; and r) increased ventricular dilation. In addition, cardiac disorders can be treated or prevented that are based on unwanted alterations in diastolic and/or systolic function, chamber geometry, blood flow, and myocardial tissue movement, such as those assessed by cardiac echocardiography examination.

Other cardiac-related responses are described herein and are also contemplated as being related to chemokine receptor activity. For example, one or more of the following cardiac-related phenotypes can be analyzed: hypertrophy; morphology, such as interventricular septal hypertrophy; left ventricular-end systolic maximum dP/dt or end-diastolic dimension; papillary muscle dimension; left-ventricular outflow tract obstruction; midventricular hypertrophy; apical hypertrophy; asymmetrical hypertrophy; concentric enlarged ventricular mass; eccentric enlarged ventricular mass; sarcomere structure; myofibril function; receptor expression; heart rate; ventricular systolic pressure; ventricular diastolic pressure; aortic systolic pressure; aortic diastolic pressure; contractility; interstitial fibrosis; cardiomyocyte disarray; Ca²⁺ sensitivity; Ca²⁺ release; Ca²⁺ uptake; catecholine sensitivity; α-adrenergic sensitivity; β-adrenergic sensitivity; dobutamine sensitivity; thyroxine sensitivity; angiotensin-converting enzyme inhibitor sensitivity; amiodarone sensitivity; lidocaine sensitivity; glycoprotein receptor antagonist sensitivity; anabolic steroid sensitivity; carnitine transport irregularities; left ventricular dilation, reduced left ventricular ejection fraction; left atrial dilatation; diuretic sensitivity; volemia; ischemia; leukocyte flow properties; the polymorphonuclear leukocyte (PMN) membrane fluidity; PMN cytosolic Ca²⁺ content; high interventricular septal defects, rosette inhibition effect; contractile force transmission; myocardial fiber disarray; increased chamber stiffness; impaired relaxation; small-vessel disease; dyspnea; angina; presyncope; tachycardia; syncope; lethargy; respiratory distress; ruffled fur; hunched posture; peripheral edema; ascites; hepatomegaly; edematous lung; cardiomegaly; organized thrombi formation; heart weight/body weight ratio; rate of pressure development, rate of pressure fail, cell twitch measurement and the like. See, for example, Braunwald et al. (2002) Circ. 106:1312-1316; Wigle et al. (1995) Circ. 92:1680-1692; and Pi and Walker (2000) Am. J. Physiol. Heart Circ. Physiol. 279:H26-H34; hereby incorporated by reference in their entirety.

Methods for measuring such cardiomyopathic phenotypes are described herein and are well-known in the art. Exemplary methods include, but are not limited to, trans-thoracic echocardiography, transesophageal echocardiography, exercise tests, urine/catecholamine analysis, EIAs, light microscopy, heart catheterization, dynamic electrocardiography, Langendorff hanging heart preparation, working heart preparation, MRI, multiplex RT-PCR, positron emission tomography, angiography, magnetic resonance spin echo, short-axis MRI scanning, Doppler velocity recordings, Doppler color flow imaging, stress thallium studies, cardiac ultrasound, chest X-ray, oxygen consumption test, electrophysiological studies, auscultation, scanning EM, gravimetric analysis, hematoxylin and eosin staining, skinned fiber analysis, transmission electron microscopy, immunofluorescent analysis, trichrome staining, Masson's trichrome staining, Von Kossa staining, 2-D echocardiography, cardiotocography, baseline M-mode echocardiography, and myocardial lactate production assays. See, for example, Braz et al. (2002) J. Cell. Biol. 156:905-919; Braunwald et al. (2002) Circ. 106:1312-1316; Sohal et al. (2001) Circ. Res. 89:20-25; Nagueh et al. (2000) Circ. 102:1346-1350; Sanbe et al. (2001) J. Biol. Chem. 276:32682-32686; Sanbe et al. (1999) J. Biol. Chem. 274:21085-21094; Wigle et al. (1995) Circ. 92:1680-1692; Pi and Walker (2000) Am. J. Physiol. Heart Circ. Physiol. 279:H26-H34; and Wang et al. (2001) Am. J. Physiol. Heart Circ. Physiol. 269:H90-H98 hereby incorporated by reference in their entirety.

For example, cardiac contractility can be analyzed. Assays to determine cardiac contractility are known in the art and include, but are not limited to, shortening assays, peak shortening, time to peak, time to ½ maximal relaxation, contracting and relaxing rate assays, changes in cardiac chronotropy, changes in cardiac lusitropy, and gross heart contraction assays. Altered cardiac-preferred expression of chemokine ligand and/or chemokine receptors, for example, can result in altered (e.g., acutely increased) cardiac contractility. An acute modulation or alteration can begin within 1 second; 10 seconds; 30 seconds; 1, 2, 3, 4, 5, 6, 7, 8, 9, 10, 11, 12, 13, 14, 15, 20, 25, 30, 35, 40, 45, 50, 55, 60 minutes; 2, 3, 4, 5, 6, 7, 8, 9, 10, 11, 12, 13, 14, 15, 16, 17, 18, 19, 20, 21, 22, 23, 24 hours; 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after administration of the chemokine ligand and/or chemokine receptor modulating agent. The duration of the modulation ranges from short durations such as, but not limited to, nanosecond, second, and minute increments; intermediate durations such as, but not limited to, hour, day, and week increments; to long durations such as, but not limited to, month and year increments, up to and including the recipient's lifespan.

In methods examining progression of cardiac-related disorders, additional criteria for evaluation can include, but are not limited to, β-receptor number, β-receptor coupling, adenylyl cyclase activity, cAMP levels at rest, cAMP levels after forskolin administration, PKA activity, PKA protein levels, troponin I (cTNI) levels, phosphatase PP2A levels, myosin binding protein C (myBP-C) levels, L-type calcium channel current density, SERCA2a protein levels, and phospholamban mRNA levels, or phospholamban phosphorylation of proteins.

10. Methods of Evaluating Neurological Responses

In some aspects, the present invention requires the analysis of neurological responses. Without limitation, a representative list of cardiac-related responses to be analyzed include one or more of a) increased macrophage activation and/or infiltration in the central nervous system (CNS) or peripheral nervous system (PNS); b) increased amyloid precursor protein biomarker expression and/or activity in the CNS or PNS; c) increased lesion formation in the CNS; d) decreased neurite growth in the CNS or PNS; e) increased neuronal cell death in the CNS or PNS; and f) increased neural inflammation in the CNS or PNS.

Other neurological responses are described herein and are also contemplated as being related to chemokine receptor activity. For example, one or more of the following cardiac-related phenotypes can be analyzed: (1) survival time of neurons in culture; (2) sprouting of neurons in culture or in vivo; (3) production of a neuron-associated molecule in culture or in vivo, e.g., choline acetyltransferase or acetylcholinesterase with respect to motor neurons; or (4) symptoms of neuron dysfunction in vivo; (5) neuronal survival; (6) synapse formation; (7) conductance; and (8) neural differentiation. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post-polio syndrome, and Hereditary Motorsensory Neuropathy (Charcot-Marie-Tooth Disease). In addition, neurodegenerative disease states and/or behavioral disorders. Such neurodegenerative disease states and/or behavioral disorders include, but are not limited to, Alzheimer's Disease, Parkinson's Disease, Huntington's Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception, can also be treated or prevented.

In combination with the cardiac disorder effects described herein, the methods of the present invention may be useful in protecting neural cells from diseases, damage, disorders, or injury, associated with cerebrovascular disorders including, but not limited to, carotid artery diseases (e.g., carotid artery thrombosis, carotid stenosis, or Moyamoya Disease), cerebral amyloid angiopathy, cerebral aneurysm, cerebral anoxia, cerebral arteriosclerosis, cerebral arteriovenous malformations, cerebral artery diseases, cerebral embolism and thrombosis (e.g., carotid artery thrombosis, sinus thrombosis, or Wallenberg's Syndrome), cerebral hemorrhage (e.g., epidural or subdural hematoma, or subarachnoid hemorrhage), cerebral infarction, cerebral ischemia (e.g., transient cerebral ischemia, Subclavian Steal Syndrome, or vertebrobasilar insufficiency), vascular dementia (e.g., multi-infarct), leukomalacia, periventricular, and vascular headache (e.g., cluster headache or migraines), as well as metabolic brain diseases which includes phenylketonuria such as maternal phenylketonuria, pyruvate carboxylase deficiency, pyruvate dehydrogenase complex deficiency, Wernicke's Encephalopathy, brain edema, brain neoplasms such as cerebellar neoplasms which include infratentorial neoplasms, cerebral ventricle neoplasms such as choroid plexus neoplasms, hypothalamic neoplasms, supratentorial neoplasms, canavan disease, cerebellar diseases such as cerebellar ataxia which include spinocerebellar degeneration such as ataxia telangiectasia, cerebellar dyssynergia, Friederich's Ataxia, Machado-Joseph Disease, olivopontocerebellar atrophy, cerebellar neoplasms such as infratentorial neoplasms, diffuse cerebral sclerosis such as encephalitis periaxialis, globoid cell leukodystrophy, metachromatic leukodystrophy and subacute sclerosing panencephalitis. Similarly, cerebrovascular disorders (such as carotid artery diseases which include carotid artery thrombosis, carotid stenosis and Moyamoya Disease), cerebral amyloid angiopathy, cerebral aneurysm, cerebral anoxia, cerebral arteriosclerosis, cerebral arteriovenous malformations, cerebral artery diseases, cerebral embolism and thrombosis such as carotid artery thrombosis, sinus thrombosis and Wallenberg's Syndrome, cerebral hemorrhage such as epidural hematoma, subdural hematoma and subarachnoid hemorrhage, cerebral infarction, cerebral ischemia such as transient cerebral ischemia, Subclavian Steal Syndrome and vertebrobasilar insufficiency, vascular dementia such as multi-infarct dementia, periventricular leukomalacia, vascular headache such as cluster headache and migraine, are further contemplated.

Finally, additional neurological disorders which can be prevented or treated according to the present invention include dementia such as AIDS Dementia Complex, presenile dementia such as Alzheimer's Disease and Creutzfeldt-Jakob Syndrome, senile dementia such as Alzheimer's Disease and progressive supranuclear palsy, vascular dementia such as multi-infarct dementia, encephalitis which include encephalitis periaxialis, viral encephalitis such as epidemic encephalitis, Japanese Encephalitis, St. Louis Encephalitis, tick-borne encephalitis and West Nile Fever, acute disseminated encephalomyelitis, meningoencephalitis such as uveomeningoencephalitic syndrome, Postencephalitic Parkinson Disease and subacute sclerosing panencephalitis, encephalomalacia such as periventricular leukomalacia, epilepsy such as generalized epilepsy which includes infantile spasms, absence epilepsy, myoclonic epilepsy which includes MERRF Syndrome, tonic-clonic epilepsy, partial epilepsy such as complex partial epilepsy, frontal lobe epilepsy and temporal lobe epilepsy, post-traumatic epilepsy, status epilepticus such as Epilepsia Partialis Continua, and Hallervorden-Spatz Syndrome.

### EXEMPLIFICATION

This invention is further illustrated by the following examples, which should not be construed as limiting.

### Example 1

### CCL5 Binding to CCR5 on Cardiomyocytes Impairs Cardiomyocyte Contractility

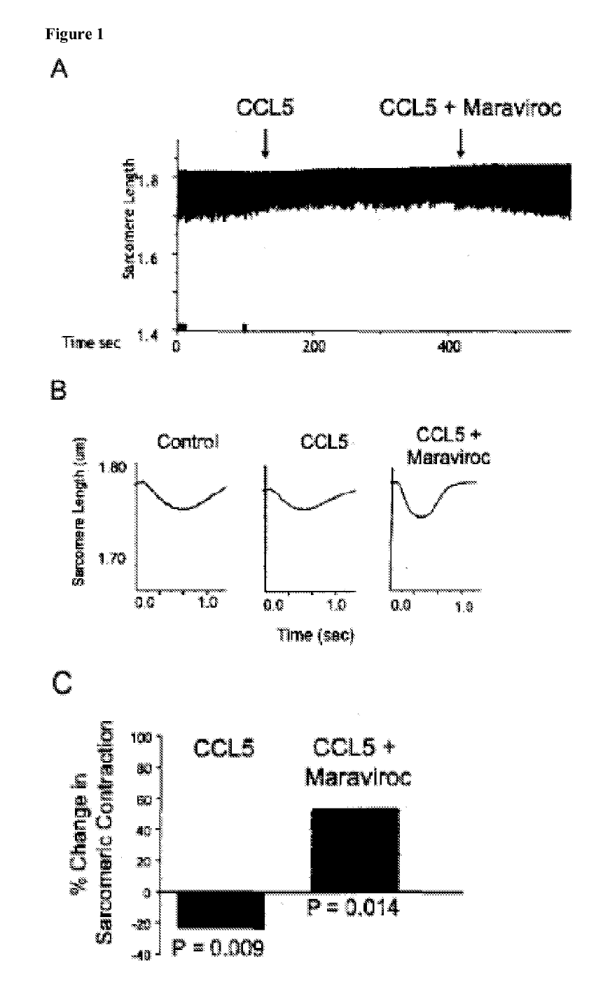

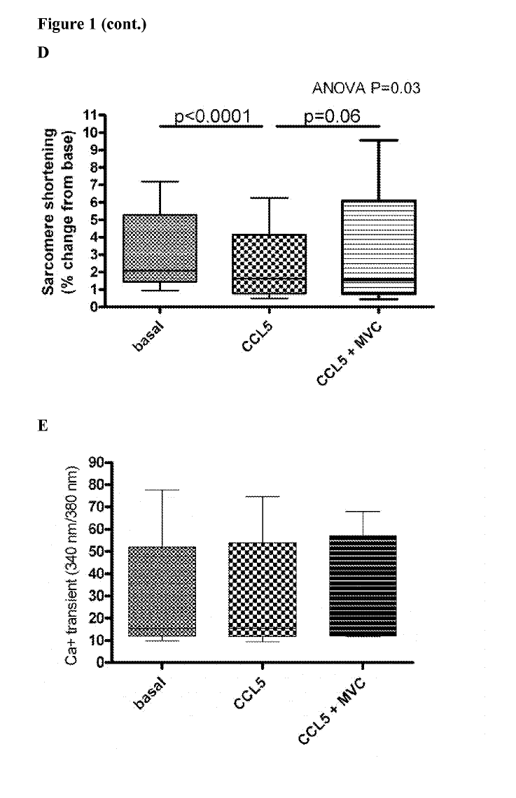

It has been observed that functional cardiac impairment develops in SIV-infected macaques. In addition to SIV, the chemokines CCL3, CCL4, and CCL5 can also bind to CCR5 and initiate signaling pathways. In order to evaluate the ability of CCL5 to alter sarcomeric contraction in a CCR5-dependent manner, isolated single cardiomyocytes were field stimulated (1 Hz) and sarcomeric contraction measured over time. Similar to SIV, addition of CCL5 led to decreased sarcomeric contraction in isolated macaque cardiomyocytes (FIG. 1A; n=4 single cardiomyocyte recordings). This reduction in contraction induced by SIV was reversed by addition of the CCR5 inhibitor, maraviroc. A representative single twitch trace of sarcomeric contraction (FIG. 1B) illustrates the decrease in sarcomere contraction induced by CCL5 (flattened curve) and then reversed by addition of maraviroc. The mean % decline in contraction induced by CCL5 was 22% from baseline contraction length (FIG. 1C; P=0.009, t-test). Maraviroc (MVC) addition reversed this SIV effect (P=0.36, t-test). CCL5 significantly decreased sarcomeric shortening (FIG. 1D; p<0.001, paired t-test comparing recordings from 14 VCM, mean of 20 cell contraction cycles/cell at steady state). In the same cells, subsequent addition of marviroc modulated the CCL5 effect towards basal contraction. There was no significant difference in shortening between baseline and MVC treatment (P=0.72). Calcium transient amplitude was not altered by RANTES or RANTES+MVC treatment (FIG. 1E; ANOVA P=0.72).

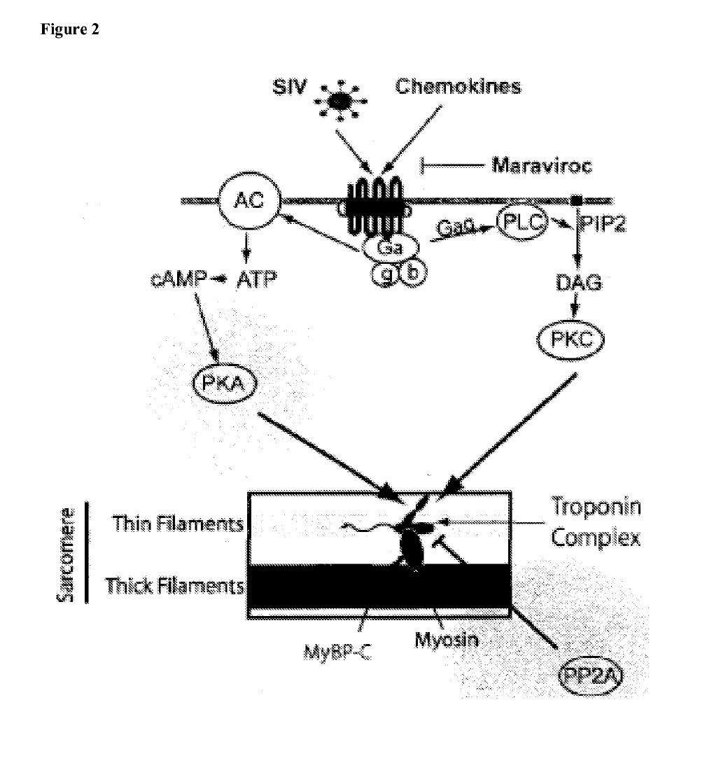

These data indicate that, like SIV, CCL5 decreases contractility. More broadly, SIV gp120 and chemokines bind CCR5 on cardiomyocytes, inducing alterations of protein kinase pathways leading to differential phosphorylation of regulatory contractile proteins (FIG. 2). These changes in regulatory contractile proteins are the molecular basis for the altered relaxation properties of cardiomyocytes.

### Example 2

### CCR5 Inhibition Modulates Cardiac Viral Load, Preserves Diastolic Function, and Improves Cardiac Dysfunction

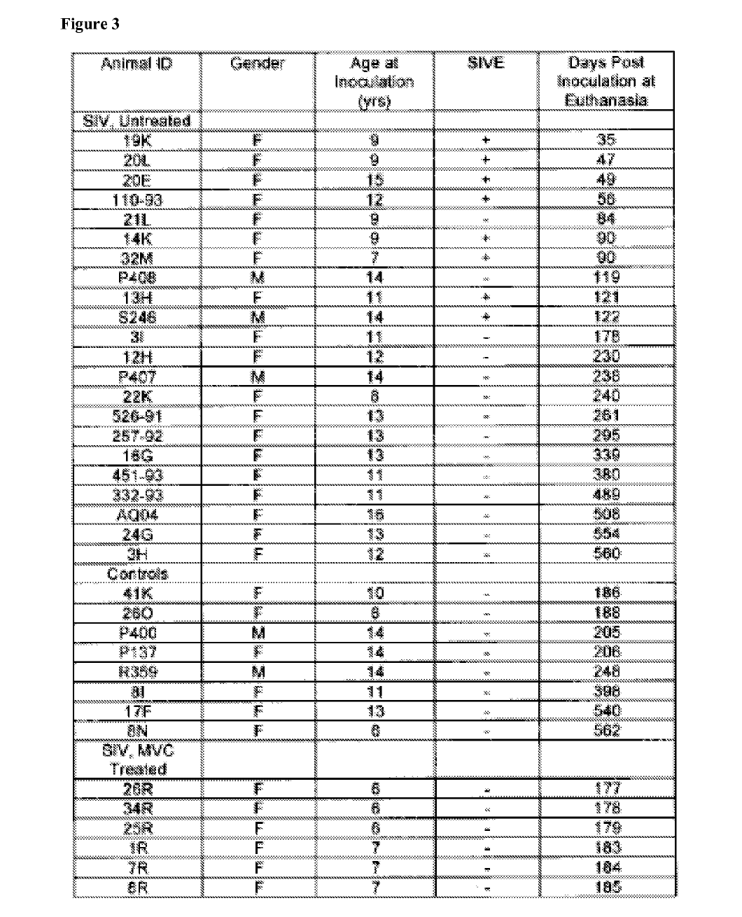

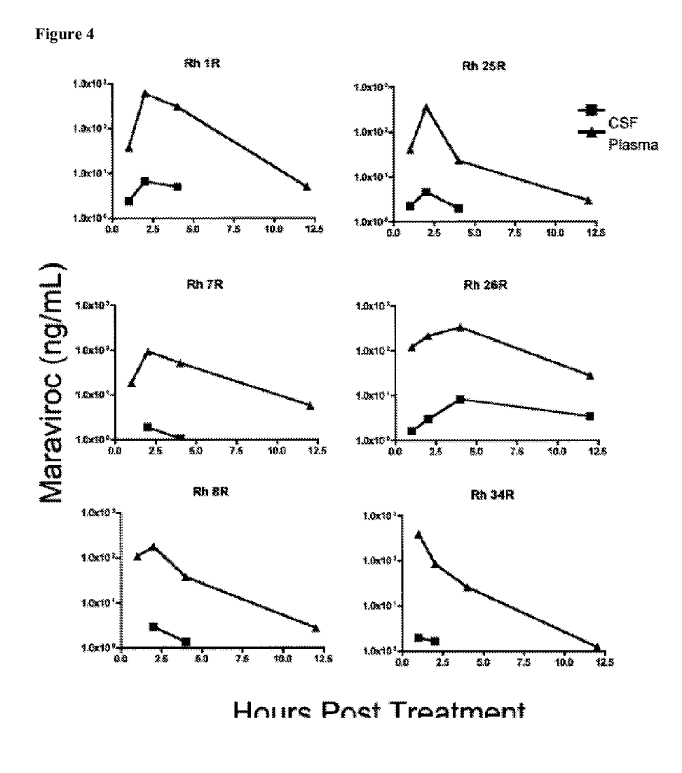

Twenty-two adult rhesus macaques (mean age=11 years) pre-screened for SIV, STLV-1, and simian type D retrovirus were inoculated intravenously with the macrophagetropic clone SIV/17E-Fr and the immunosuppressive swarm SIV/DeltaB670 (FIG. 3). Age-matched adult rhesus macaques (mean age=11 years) were inoculated with media alone as controls. SIV-infected animals were euthanized when two or more AIDS-defining criteria were observed (mean length of infection=218 days postinoculation); control animals were euthanized at time-points exceeding the mean time post-inoculation for SIV-infected animals to control for potential confounding effects of ageing on cardiac performance (mean length=317 days post shaminoculation). At euthanasia, all animals were perfused with saline to remove blood from the systemic vasculature. Hearts were immediately harvested and samples were immersion fixed in Streck Tissue Fixative (Streck, Omaha, Nebr.) or flash frozen.

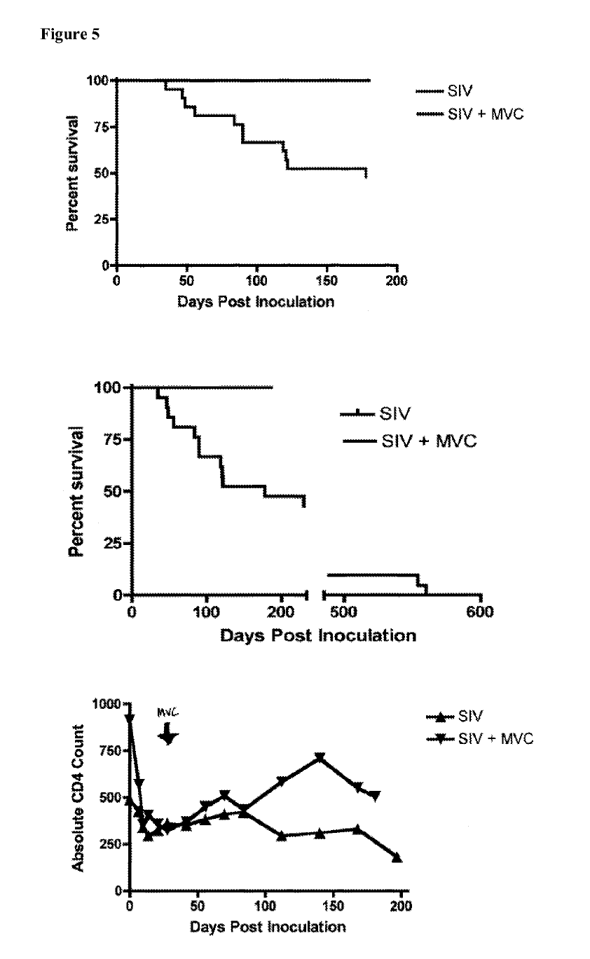

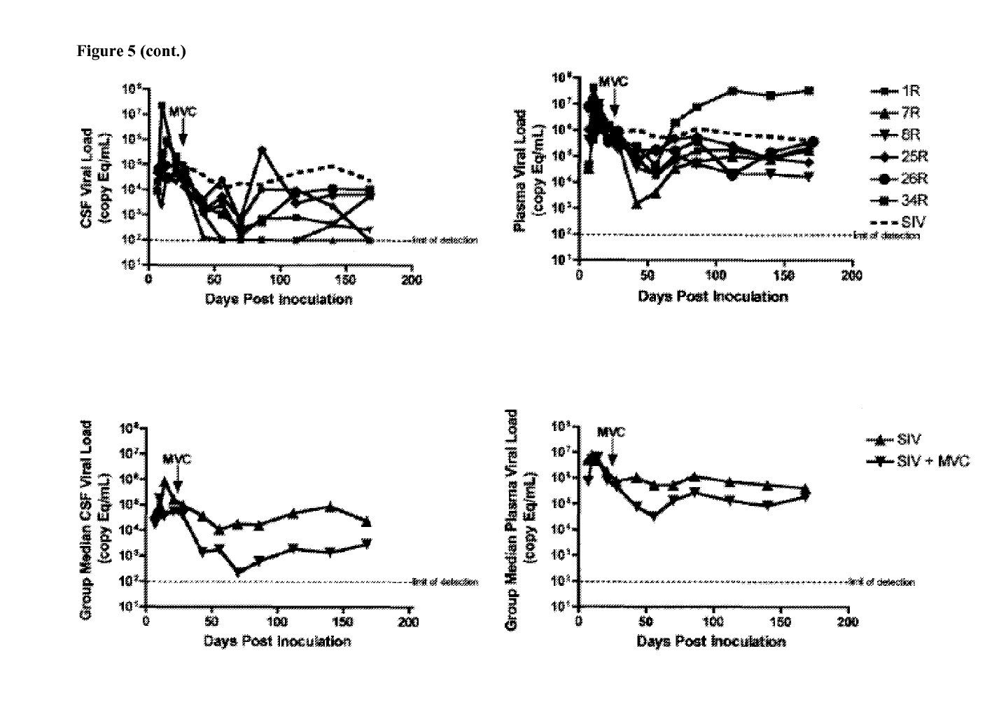

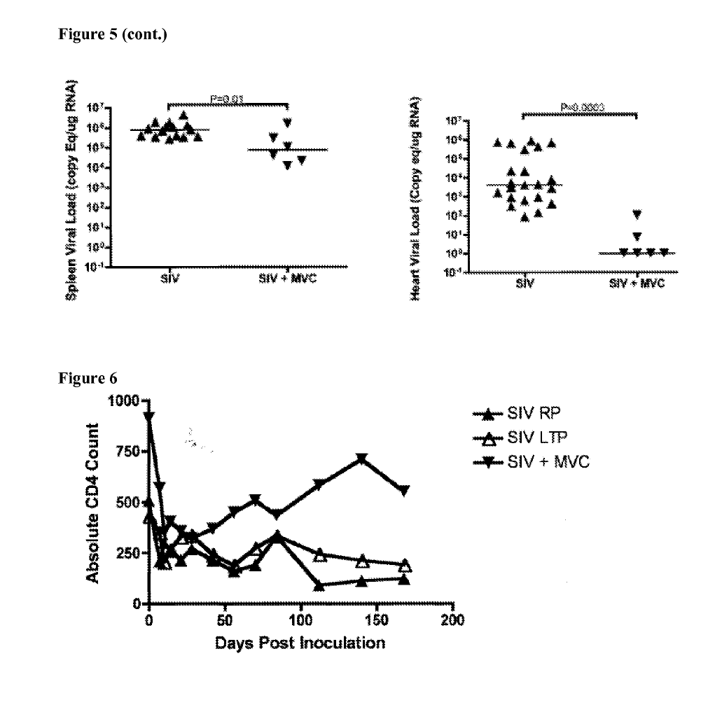

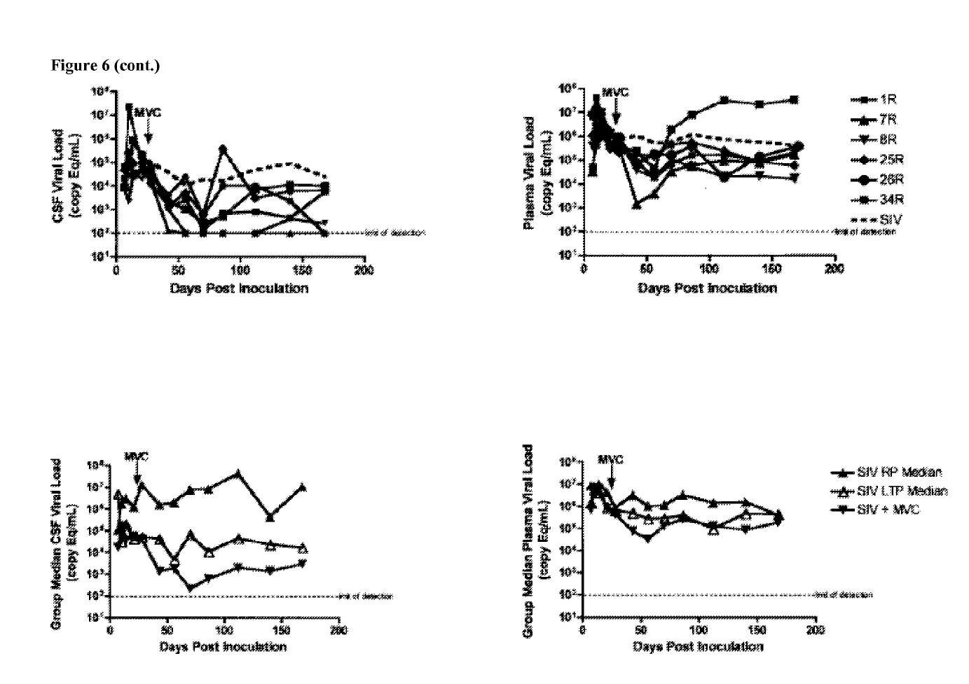

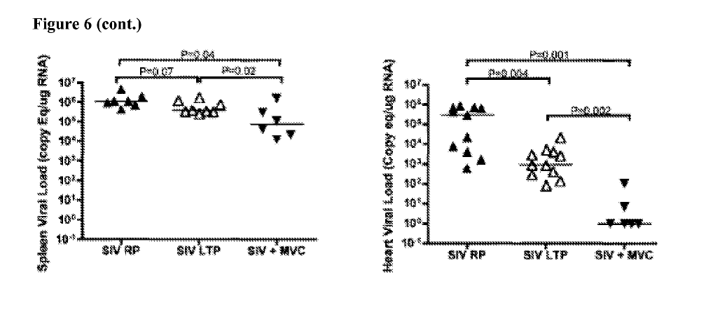

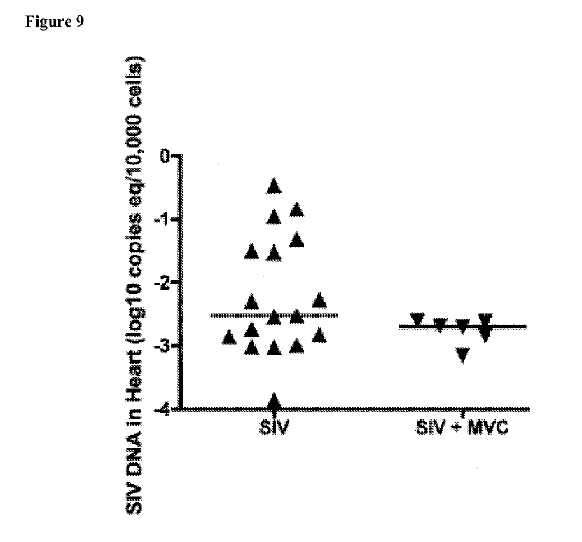

Maraviroc was administered to a cohort of 6 macaques at a dose of 40 mg/kg orally twice per day and the bioavailbility of the treatment in cerebrospinal fluid (CSF) and plasma is shown in FIG. 4. The maraviroc-treated macaques showed a significantly increased survival relative to the untreated cohort (FIG. 5), which was accompanied by increased CD4⁺ cell counts, as well as reduced viral loads in CSF, plasma, spleen, and heart (FIGS. 5, 6, and 9).

Prior to inoculation, all animals were evaluated by pulsed-wave Doppler (PW) and Mmode echocardiogram in combination with 2D echocardiogram to record average baseline myocardial velocities, wall thickness and chamber dimensions under ketamine anesthesia (5-10 mg/kg IM). Three pre-inoculation studies were performed on each animal to establish individual baseline values. Echocardiography evaluation was repeated immediately prior to euthanasia. Trans-thoracic echocardiography was performed in the left lateral decubital position using a Sequoia Acuson C256 ultrasound machine (Malvern, Pa.) equipped with 15 MHz linear transducer and ECG monitor. Data was recorded over five cardiac cycles at a sweep speed of 200 mm/s with simultaneous three-limb lead electrocardiography stored digitally and measured (mean of 3-5 values) at the completion of each study. The heart was imaged in the 2D mode in the standard parasternal short, parasternal long, four and five chamber axis views. Left ventricular diastolic filling patterns were determined using the mitral inflow and pulsed-wave tissue Doppler analysis. Mitral inflow and left ventricular outflow velocity-time interval traces were obtained by placing the pulsed Doppler sample adjacent to the anterior mitral valve leaflet tip in the left ventricle outflow tract in the apical four-chamber view. From the apical fourchamber view, the sample volume was placed in the ventricular myocardium immediately adjacent to the mitral annulus in either the septal or lateral wall. Chamber dysfunction was established using ejection phase criteria, including ejection fraction and mitral systolic annular velocity. Diastolic function from e-wave deceleration and isovolumic relaxation.

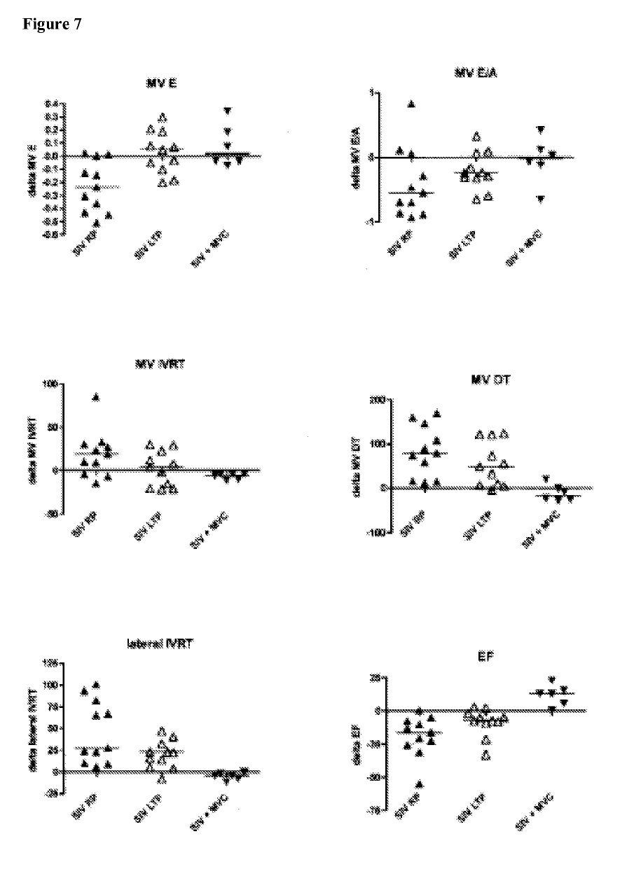

Cardiac function in SIV-infected and uninfected age-matched control macaques was evaluated. Alterations in cardiac performance were calculated by subtracting terminal time-point measurements from the mean baseline values for each individual animal. SIV-infected macaques had significant changes in multiple mitral inflow parameters consistent with diastolic dysfunction with impaired relaxation (FIG. 7). The parameters were significantly ameliorated by treatment with marviroc (FIG. 7).

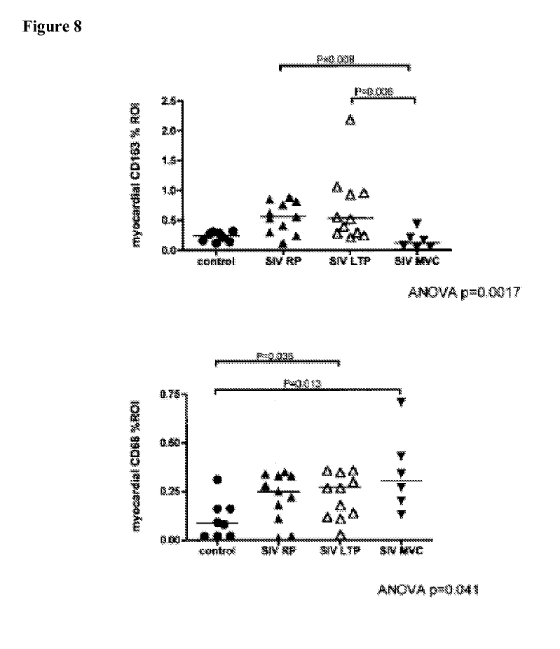

To characterize the mononuclear cell populations in myocardium, sections were immunostained for the macrophage marker CD68 and measured by quantitative image analysis. In uninfected animals, CD68+ immunostaining localized to single, round cells located diffusely throughout the myocardium with increased numbers around vessels, consistent with resident macrophages. In SIV-infected animals, CD68+macrophages in perivascular areas were more prominent, likely reflecting recruited, infiltrating macrophages. Amount of immunostaining for area occupied by CD68+ cells in the myocardium was significantly higher in SIV-infected macaques (FIG. 8). Myocardium was also immunostained for the macrophage marker CD 163. Studies in rodent models have demonstrated that ED2, the murine homologue of CD 163, is expressed on a resident population of myocardial macrophages. In control animals, spindle shaped CD163+ cells were present diffusely throughout the myocardium occasionally forming small clusters. In SIV-infected macaques, cells were similarly spindle shaped to stellate with more extensive cellular processes. Immunostaining patterns demonstrated a similar population of resident macrophages in the rhesus myocardium. Total immunostaining for CD 163+ cells myocardium was significantly higher in SIV infected macaques versus control animals (FIG. 8). Myocardial CD163 immunostaining was highly correlated with CD68 immunostaining.

### Example 3

### CCR5 Inhibition Modulates Neural Viral Load and Provides neuroprotective effects

To determine whether the CCR5 antagonist maraviroc altered CNS disease progression, SIV-infected animals from were treated with maraviroc and CNS outcomes were compared with untreated SIV-infected animals. Specifically, six rhesus macaques were inoculated with SIV/17E-Fr and SIV/DeltaB670 and treated with maraviroc (200 mg PO BID) beginning 24 days post-inoculation until the study endpoint 180 days postinoculation, 22 SIV-infected animals served as untreated, SIV-infected controls, and 8 additional animals served as untreated, uninfected controls (Example 2). Maraviroc levels in plasma and CSF were measured using liquid chromatography tandem mass spectrometry. SIV RNA levels in plasma, CSF, spleen, and brain were measured by qRT-PCR. Immunostaining for CD68 and amyloid precursor protein (APP) in the brain was measured by digital image analysis. Group comparisons were performed by the Mann-Whitney test.

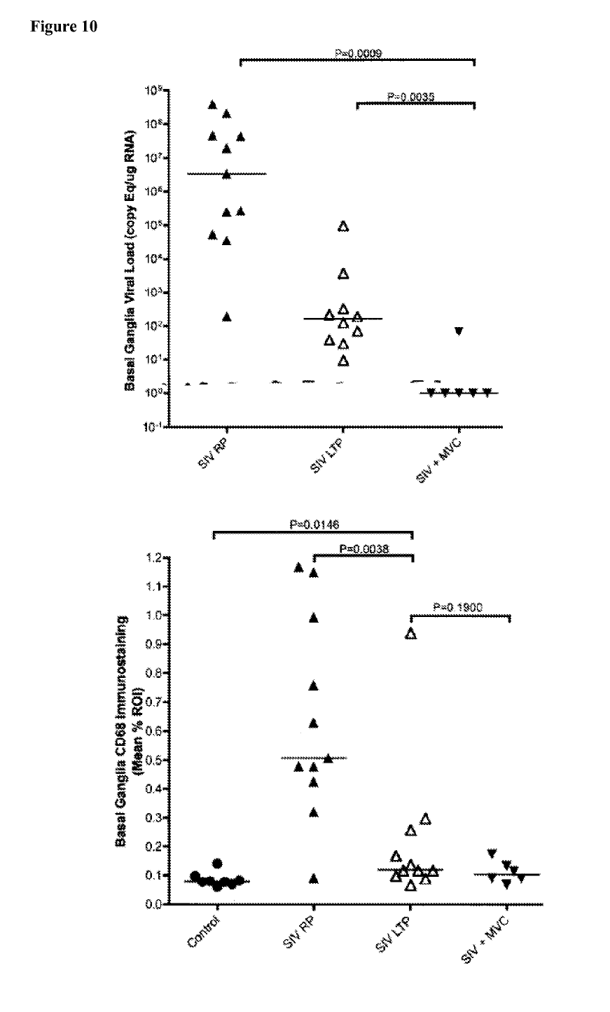

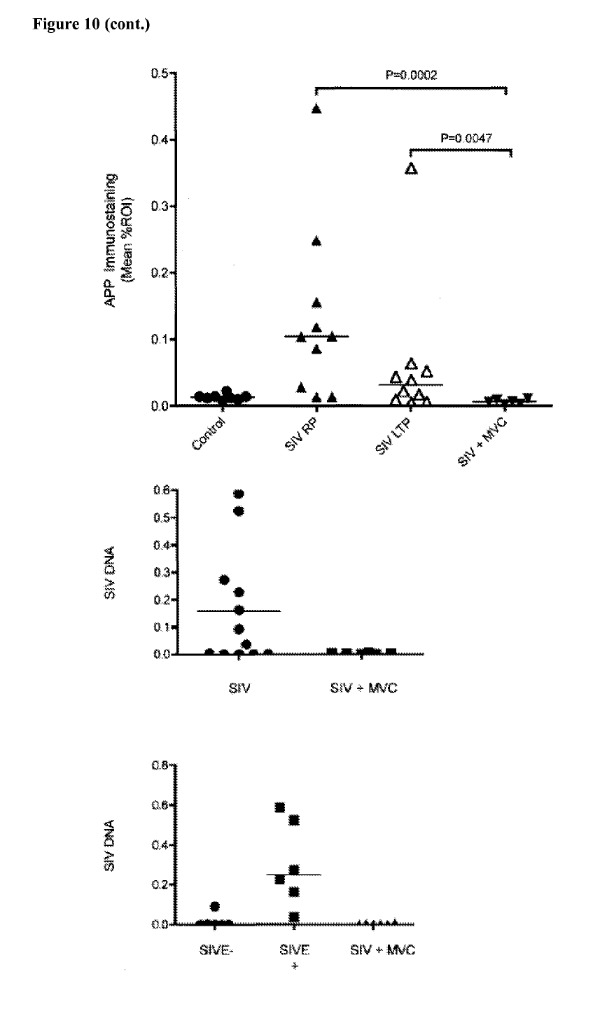

SIV RNA levels in CSF and brain were significantly lower in maraviroc treated, SIV-infected macaques versus untreated SIV-infected macaques, demonstrating that maraviroc monotherapy limits replication of SIV in the CNS. In addition, maraviroc treatment lowered CNS macrophage activation represented by CD68 immunostaining (P<0.001) and axonal APP immunostaining (P=0.003) to levels present in uninfected animals (FIG. 10). Although maraviroc therapy also reduced plasma viral load and SIV RNA levels in spleen, relative decreases were less substantial than CNS declines, underscoring the need to focus on CNS-specific outcomes in evaluating efficacy of CCR5 inhibition.

Thus, maraviroc monotherapy significantly improved SIV CNS disease outcomes with marked reduction of SIV RNA levels in brain, reduced cellular immune responses, and less neuronal damage.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing a cardiac disorder in a subject comprising administering to the subject an effective amount of at least one antagonist of a chemokine receptor expressed by cardiomyocytes and/or inflammatory cells in the myocardium of the subject, wherein binding of the chemokine receptor antagonist reduces binding of at least one chemokine to the chemokine receptor, thereby treating or preventing the cardiac disorder in the subject.

2. The method of claim 1, wherein the subject is infected with at least one primate immunodeficiency virus (PIV).

3. The method of claim 1, wherein the subject is not infected with a PIV.

4. The method of claim 2, wherein the PIV is selected from the group consisting of simian immunodeficiency virus (SIV), human immunodeficiency virus 1 (HIV-1), and human immunodeficiency virus 2 (HIV-2).

5. The method of claim 1, wherein the chemokine receptor is selected from the group consisting of chemokine receptor 5 (CCR5), a receptor that can bind chemokine ligand CCL3, a receptor that can bind chemokine ligand CCL4, a receptor that can bind chemokine ligand CCL5, a chemokine receptor that can bind SIV, a chemokine receptor that can bind HIV-1, and a chemokine receptor that can bind HIV-2.

6. The method of claim 1, wherein the chemokine receptor antagonist is selected from the group consisting of a nucleic acid, a peptide, a peptidomimetic, an antibody, and a small molecule that binds the chemokine receptor or nucleic acid encoding the chemokine receptor.

7. The method of claim 6, wherein the chemokine receptor antagonist is a small molecule selected from the group consisting of 4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide, vicriviroc, NCB-9471, PRO-140, CCR5 mAb004, 8-[4-(2-butoxyethoxy)phenyl]-1-isobutyl-N-[4-[[(1-propyl-1H-imadazol-5-yl-)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicy-clo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carbox-ylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carb-oxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide), Sch-C, Sch-D, TAK-220, PRO-140, or a pharmaceutically acceptable salt or solvate thereof.

8. (canceled)

9. The method of claim 1, wherein the cardiac disorder is selected from the group consisting of myocarditis, dilated cardiomyopathy, left ventricular dysfunction, atherosclerosis, coronary artery disease, coronary heart disease, coronary vascular disease, peripheral vascular disease, myocardial infarction, and heart failure.

10. The method of claim 1, wherein the cardiac disorder is selected from the group consisting of a) decreased cardiomyocyte contractility; b) decreased left ventricular ejection fraction and/or volume; c) increased end-systolic and/or end-diastolic fractional shortening; d) decreased mitral valve annular velocity; e) decreased mitral inflow; f) decreased aortic velocity time integral; g) decreased aorta cross section area; h) decreased isovolumetric contraction time; i) increased isovolumetric relaxation time; j) decreased heart chamber mechanical efficiency; k) increased cardiac protein kinase A and/or protein kinase C biomarker expression or activity; l) decreased phosphorylation of cardiac myofilament protein biomarker; m) increased macrophage activation and/or infiltration in the myocardium; n) increased chemokine biomarker expression and/or activity in the myocardium; o) increased cytokine biomarker expression and/or activity in the myocardium; p) increased cardiac fibrosis; q) increased cardiac inflammation; and r) increased ventricular dilation.

11. The method of claim 7, wherein the cardiac disorder is determined by assessing a) cardiomyocyte contractility; b) left ventricular ejection fraction and/or volume; c) end-systolic and/or end-diastolic fractional shortening; d) mitral valve annular velocity; e) mitral inflow; f) aortic velocity time integral; g) aorta cross section area; h) isovolumetric contraction time; i) isovolumetric relaxation time; j) heart chamber mechanical efficiency; k) cardiac protein kinase A and/or protein kinase C biomarker expression or activity; l) phosphorylation of a cardiac myofilament protein biomarker; m) macrophage activation and/or infiltration in the myocardium; n) chemokine biomarker expression and/or activity in the myocardium; o) cytokine biomarker expression and/or activity in the myocardium; p) cardiac fibrosis; q) cardiac inflammation; or r) ventricular dilation in the subject or relative to a baseline.

12. The method of claim 10, wherein the expression of the biomarker is assessed by detecting the presence in the sample of a protein corresponding to the biomarker.

13-20. (canceled)

21. A method of treating or preventing a neurological disorder in a subject comprising administering to the subject an effective amount of at least one antagonist of a chemokine receptor expressed by neurons of the subject, wherein binding of the chemokine receptor antagonist reduces binding of at least one chemokine to the chemokine receptor, thereby treating or preventing the neurological disorder in the subject.

22. The method of claim 21, wherein the subject is infected with at least one primate immunodeficiency virus (PIV).

23-24. (canceled)

25. The method of claim 21, wherein the chemokine receptor is selected from the group consisting of chemokine receptor 5 (CCR5), a receptor that can bind chemokine ligand CCL3, a receptor that can bind chemokine ligand CCL4, a receptor that can bind chemokine ligand CCL5, a chemokine receptor that can bind SIV, a chemokine receptor that can bind HIV-1, and a chemokine receptor that can bind HIV-2.

26. The method of claim 21, wherein the chemokine receptor antagonist is selected from the group consisting of a nucleic acid, a peptide, a peptidomimetic, an antibody, and a small molecule that binds the chemokine receptor or nucleic acid encoding the chemokine receptor.

27. The method of claim 26, wherein the chemokine receptor antagonist is a small molecule selected from the group consisting of 4,4-difluoro-N-{(1S)-3-[exo-3-(3-isopropyl-5-methyl-4H-1,2,4-triazol-4-yl)-8-azabicyclo[3.2.1]oct-5-yl]-1-phenylpropyl}cyclohexanecarboxamide, vicriviroc, NCB-9471, PRO-140, CCR5 mAb004, 8-[4-(2-butoxyethoxy)phenyl]-1-isobutyl-N-[4-[[(1-propyl-1H-imadazol-5-yl-)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicy-clo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carbox-ylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carb-oxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide), Sch-C, Sch-D, TAK-220, PRO-140, or a pharmaceutically acceptable salt or solvate thereof.

28. (canceled)

29. The method of claim 21, wherein the neurological disorder is selected from the group consisting of Alzheimer's disease, Parkinson's disease, Huntington's disease, Pick's disease, Kufs disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy(LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

30. The method of claim 21, wherein the neurological disorder is selected from the group consisting of a) increased macrophage activation and/or infiltration in the central nervous system (CNS) or peripheral nervous system (PNS); b) increased amyloid precursor protein biomarker expression and/or activity in the CNS or PNS; c) increased lesion formation in the CNS; d) decreased neurite growth in the CNS or PNS; e) increased neuronal cell death in the CNS or PNS; and f) increased neural inflammation in the CNS or PNS.

31-38. (canceled)

39. The method of claim 21, further comprising administering one or more agents that inhibit the neurological disorder.

40. (canceled)
